SEQUENCE LISTING (18 sequences)

SEQ ID NO: 1 - DNA, 10113 bp, Homo sapiens
ctcgagtcagtgtctggcagtgaatgagctacaaattgttttcatattgcttacctgaaggccagtgcttgtttagctgctgaagaaaaatagaaaccttatggcatttagaacatagtttattctttaagtgcagaagtgtgtgacttaacccttgactggcatggtcttagctcctgtttacaatttggtatcttactgccacaaagagtctgttctatcagtcttacattctctattttcacatcaatgctggccagttgtgtctaaacactgcaaaagggagggtatataacaagatatgtctgacttcctgagccatcatggctgggaactcagtttttaagatttatctagggtccatttggccaagattggggtggggagggtctgttcagtcagttgagggctttaagatttatttttagtttacaggaggaaccatgttcagactaccaaatagtttccaatgctgagcagggctagggccacagacacacactcctgtttccatagaaattcctcaggcaggcctcatgtacttagccaggaccctgctcttgcccccaaaggaatttatccttttaaaagtataatattcctcttatctgactgttctactgcacagtggggtaaccacagttaacaatatcatatactatatttcaaaatagctattaatagaagagagaattttgaatattctcaccataaagaaatgacaaatgtttgaagtgattgatatgctaattaccctgatttgatcattacataatgtaaacatgtatagaaacatcacttaaatataaacatgcacaattactagatgtcagttaaaaacaaaataaaacttagaaaagtataatgtgatgatactggttcagggatctgatctttaatccgaaggaaaagaggagacatgaaagaactgaggggagggcttgttattcctcttcctcccctttctctattaccacccttgcattaacatgcccctggttatcttgctatgttttttatatttattatgaatgcagaacacagcaaaaagtaaatagcaattatatcagttctaattctgcttcaagtaatagaacaactcaaagcttattaaattaaagggagaatgtattggttcacgtaattgaaaagtcttgaaatggggctagcaggctgtactcactccaaggctcaaagtatatcatcaagattgggtgagtctctcaatctctcagctctgcttttccctctgttgactacagttttgacatgtagtcatgagatggctgcagcagcttggcctacattcttccctgttcaaatctcaaggtgcaggggccaaggggttggggggagaggtggaggaggaatgctatgtctttattggtagttcacacaatagtcctgagattcactctgccttagacatcccaggtcacatgcttacccatccctgtccactaacatccaagcaacatcttcaccctcataagtcaaagataaactcaccccctcaccctgaccacaaggagtaagaatgggcaatggttggattcttaaatgaaaatcagggactgttgccagaaaaaaaaaaaagtggaatgtatacaatggaggaaaacaataaatgtactagagcaatttttgctactttttttgtagtatttgtattcaacattcaatattctcctgaagctactcttttcctaaaacagatgaaaaccttctccccgatcttctagagatgatcacactgaaccctaaaggacatttaaaaacctccataatcacacagagagggactcaattaaaaaaaattcttggaaaagaaaaaggaaaagatgtatgtttcttgctcttctctcttgatggaaacaaacacacatgcaaaaattcaattgcaataagtgtctcatattgagtttaccaattctgtaagacttcatagttatataaaactaataattaccatttactgaataccttgctatcatgctaggcaaagtgtcagaccattgacatataattcatttaatatactgaagaactatcaaaatgaagtattattatctttactctatagatgaggaaacagacccagaaaggttaagaaatttgctcaaagtcatagagctggcaactggaggagccagtagtcaaaggcaagtctgtgaggtgtgtgagattgtgctctcaacactacaccatggattttgtgatcttggctctaattagggggccaatatggtggacatggtgttatttgaatttaaccatttttcatatctcatatgaaaagttcctagaattaaaaatttcatgaagaaaacaaaaaatagctgaggatttctagatgcatactgtgaaaagaaatgcaaatattaatagtaaccatgttctatgaattagtttaagcatatacatgaaatgctgttagagtacacgttgcaagagtaactaactgatggcctgattagaaaaatgatagaatgaggtaaagaaagaggtagattctgggaaatatggattaaaatgatgaaacaaaaaatgagaaaaaaactaccttaccttctcaagtattgccatagaagagcagaagtcatcctgagcaaagctaaaggcaaaaaacctaggctccagactgatacactggcttgataatccacactgtgagatgtcaccaaaagttgtctgatccagctgtggggacttggctccaagttgtctggatatcctaaaataatataaatcccttgaaacaagtgctctaagaaatggaggaagccatactttagggaatgctgataaaatgagtggaaatgttggtggtgtgagtccacctctacctcaacaaggtcaccactgatgtgagaatccatgaggttaacaaaaacgagcatcattagtctcataaattaacacttgaaggactttggagccttgggtctcagtgattcttctggaaatatggtagtttagggtctttaataatttttttaacttttgttatgagcagaaattgtaaataacatgtaaatacaactccagagtggttctgatgttcaccttctgaactcatctagttctcaaactactagattatgcaacccttgtgctcctacagctaaaccacatatactattctgagccctcccctaactacacctgggtaagaggagtgtggtgtagtctggtgttgtgctataccaatagtcatggagatatcaatcaaacatctgcctaatctacaaggtgtaggtggtgaattatgaagaatgagtaaccaggagacttacgatgagagaataaagtctaagttgataaaaagttagtgctgagtagatagaggggtcttcaggaattggaagcttggtgagggtccccatcatccagaataagtactggtcttcagcaggaagaatttaagaatagaagttacctaaagctcatgaattagtatcagttcaattatgctggtaatctgattttgatctttctcagttatgataaaagtctctgcctggtcatgatataaaccttctttgttctttagtcttccctacttacaagattaaagtcctggcctaaaatatgagccctaaggtcctgctacctagtgacatgatattgttgataccaactctccattacccactgcctactgaaatctcctattcctgccttctccatcattgagttctgtccacctgtcagagtctacttcattgggttgccctccctaacctgcccaacaccaactgcttgccaggccgctaacctattccctcttgttctgcaccttctctcatagtccacttccaatgcatcaccaagatctgcctctaaaatgtatcttctcttcaatcccactgccaatgccttagtttgagattttactatctcttccttcttttatataaattgcctgtatccagctcaatccgccatccatacttccagtagttacatttactgaatgaaaatctggtcctgtcacttttccacttaaaaacccttcagttgccacccaatataaataatatatacatcacttagaggcttccaaggcttccaagccctttccatatgacctttcaggcctgcctctccctgatctttcccatacaatccaataagtgcttttattgttctttaatgttctttaatgtcctttaatgttcagctcaaatattacctccccttgacctctctaaaagaaaacagcttgcttcctcaagtaaagctatttactaatacctaaggtattcctaaaggtatttacaaatacctttattataacacatatcatactgtttatatgtatgctttcttcattagattataaactcttggaggacaaggagcatgccttcttcatttacagttctgtgacaagcatggtgactggcaagtagtcactggcaagaaatgtttcttgaatgaataaatgatccctaaatactgtgacctatctcttagtctgaatttccctcagttacctgcagaaattttccctctggaatatattcttgtttatctatctgtctacctgtctgtctgtctgtctattctatctatctataatctccctatatacaaaggaaacaggtgagaaaggagagtagaagcttatttcaagttccagtccctcctgatctacattctcctgtaattattagcctatgttaccatgtctgaacagaaaatattggtgatgcccttgatcatgaatagctatcatgtctctgttctggcttgctcctggatttttttttttccagtttcatgatgctgcatcactctgtcaccagtcctcactgttcgtttctatggaacatgaaatggtgaatggtccatcctttactgctgatactagtcattgctgaaacagccaccctaaagatctcacagtctctctgtttataaacatttaagagtcaagtcatgaaggctttctcccttaactacatgggatgatcagttgttttatctgttattttttctgttattacttttgtccttaattgttagagaaaactttcatataacacagttactacataaatgccccctcccttcacatcttagaatgcctctggccatctctgtagttgtacttgagaggtttaataaagtaccataagtttgggaaaattagctttattgatataagcacttcctggagaatatttcttcactatacaaaaagctcatatttgatcattgttttctctatatctgtctatctctctcacatacattctgagtgcctgtgtgtgtgtgtgtgtccccagtgacttctgcatctgtagtagctggaataacatgtgtgtcacatgtacccccaccacacacacacacacacacacacctctgccacgggcttcgttcaattctattctcaatttccaaccttgtcagccatcttgatttcctcttctccaaacctcaaagaaactgttaatgagtacccgagaatgaaaatgttgggcatatggaaaaactgaaagacaatccatattgccataaaatggcctgcttttatctggaaaagccttattatcactcacactcattccttctacatcctttacctccttctctttgtctttctgtctgtctctcattctgtctctatcacacacacacacacaacacacacagagagagagagagggagagagagagagagagagagagaaagagagactggctataaagaaagcagcatctgagggcatcaatggtaattcgaacattttgtttgcttgggatcaagattcctttccatcaccattgtcctggcaaatataagctgcaagtggaagtgttttagccagatactgctccaccactctatggctaactaagcaggaatatcagcttcaactatgccatggaattcaagagaatattcaacctggaaaaattctaaccccaaacagcacctcccaaaagatgactacagctcctgtaggaaatcatttaaccacaaattccaactccccttcactcctacagcctcagtcacacatctcaaagggctgatccttgaattgtgacaacctgacccacatcagcagcccagaggccagatggcaacacaatgcctttccagcctactgggtaggaaaagggagggacaagcaattgaatgattataattgaaatcctgtaatttattatttgtcaataactcctgccttgggggagttcccttcactccttagcaaatgctggcagccgcaatatgtgaccacagacacctaaaacaccactgaaagcatttcattatgtggcaacaatgatatggagaataagatctctctggagataagaagacatgccacatctcagagttcactattcaacagcaaagaatttgaatagggggaaaaattccttagacttagggggaaaaattcctgagattctgagggtaaaaagctagcatgcaagtgggatcagccagactggcaggaagtggggcatgaaaacccaagaactatcctcctgtttgcagtataatgttacccctgcagttattaaactgcacagatcaaataactatttgaactgagctgagctgagtcaagcaaagaaatgtacttcaatcatagaatagtagctgtcaggttgaaaagggcatctatcagacatctacccaatgttcctctacctaactcccatcctaattatccctgtctcatgttcatctccctgtaattgatgcaacctgatgcaacataaagaggatttggagtgaaaagccctgagttggaggcctgggccttgactttttaatttactagccataccacattggtccttctagtcttcagctacaatgtgagcagattaaactagttgatgccaagattccatccagttcaaaattctctaggcaataatagggaactcactatctccaaaaataattatttgcatgtttggacaattctattaaaaagtcgtcacttacataaagccaaaacatggtttcttatagtttctacatatttatctgagttctacctgccttgagattatatagtccaagtataatccctcttctatataacagccactcatatagccaaagacagatattgcatctcccatccatctatccatccatccatccatccatccatcccactgagttctgtttgccactcatctagacaatcattctatctactttactcagaacacataccaatttgtccatatccttactgaaaaatggtcatcagaactgaacaaagagctacagaaatagtttgataaatgctgagccaagtacctcaccacctattttgttccagctaatgtgtttattttaatgcagcctaatatcacattatctttttggctatggcatcactctgccactgccacaatattgctttttttactcatgtcttctaccctatccttgtaaagtgtaatttggaactggagtcagaattttactttttcaataatattcatttatcagatttgagccattggtgcaacatgataaaatcttcttgaatcctcagtctatcctaaaaatatttgccatctatccaaattttcacctaagttattgataaaatacaatgagcacaacatggtcaggaaaaaagtcctagagcagaccactaaacaataccctccatattgacataattctgcaagatttggggtcttttggccccaaagaaggtcatatccaagattgcctcattcttgacactctctcaatgctaccttcttcactcactcatagtccatggtctagtctttctgctgatcctgcaatgatttccacagttaagttcacccagacataggtactttgagagaaatcactgtttaaaacattaaaaatataaataaggttaacaaaaaaaggataaaagttcacacccagaacaatatgccatggtggaaataatgtgggagttggagtctgaataacctcagattaaatcctagttctgtaacttacttgcttggtgaccttgggtgccttatcagtatcagttccctcatctctaaaatagaggaataataatgacctcaaagggttgtcctgatgattaaaattaatacacgcaaaatcttagcacattcctagcacataatgggtatacaatatagattactatcattattacatcatggtagagacaaaaatgcaataactactggcataaaaaaattagacccaagaagaaaaaaggagaaaatgaatatctgtgttctacagatgttaaccaatactccacaaaagaaagaatcaatggtctaataaggttaagaacgaggacattaaacacagttttaaaaactcgggcatatttttatctctttgactctgaacaagattggcacacccttgcatcccttatttgatcatgtcccaggcattgctgtagggcccaaggacacaaaggatgactcagacccagtcaatgctagggtgagaagaaacacaggattcaaggcaccagggggcagggggcacacatataaataagtaattaatataaaatatcaccagtgctattgagaggtaaatacagagtatgtgagggaacactgataaatctggagatgtcaggtaaggctttatgcaggaggtggtaattttgaagaatcttaaaagataaagagaagttaaggatgagggtctctcaagtcaaggtagtggaggatagcacgcaaagattttgaacagtagtggaccaatatgggctcgtccaagaaatattgctcattctagagtgacttttccatgagatacaggtaataagagggacaattgaaaagatacagattcatgcctattgtaaaggggcttgtacgtcaggtaaaaaagtctgaaattctgcaggcaagaagaaaccagcacagcgaatgacataagatggtaagccagcttaatagtagagagagaggctagttacgacacttttttttctttaccaatacccaattgaaagatggtcaggtccagaactaaaattatagcactggaggtagagaataggcctacataacaaaatctaagcctgagtataattgatacgatgtgacatgagcatgaggggttaaggaaaaggagaaatgaaaatgactttgtctagcttgagacacccagtgtgtgggggcatcattaccaatatatgggcttctggaggatagtacattttggtaggaggcacaatgtaagttcagttttcaacactatggattgagataaccatgggacatccatatggagatgcaatctgctcaacataacagtgtgtatatcataagacagaccagggataggagtcccctgtatatttatggcaataaaagaaaattcatggtttaagaaggaagagtatgtggaacatgtctctgatgccacgatgtaataaccttgaaaacaaagtaaaattacactaatgagtcttgcctaattaaactcatgctcctagtgatcaccacttctagttcaattgttcacattcttgctctgctttgaaaaattaaaattaaatttgcctatcctctactgaccataatttctagaagacggcattcatctcatggcaagttcttcagtacccaaagatggaatacatagattaaaaaagaacatatatgtagatgcttgtgatgttttcctatcataaattgaatttcaagttcttataaacgtattaatatgtcctactcttctagagacaaggatcaggaagtgtatttatcaatagatatttaccaagcacctgtcaagccaaagtggggttacagaaaagtaggtatgggccctgcacacaaacaacctgtattagccaaagggacccttccataaaatttccaatatgtaaacccaaatttggaacttgctgaaacaagtacagatgagtacgtgaggaagctgggaagtaaacacaggttgctggagaaatagaggtggagatatgggtggatctaggtttggttaggaatgaatcagaccatcccacagagggtggctcctccctgcatggggcctgctataaaagggccattatctcagccttcagtacccagcaggctccttcaggctacattctatttgct

SEQ ID NO: 2 - DNA, 15 bp, Homo sapiens
cttttggtgaacaag

SEQ ID NO: 3 - DNA, 18 bp, Homo sapiens
gtaagaaggaatacgcgt

SEQ ID NO: 4 - DNA, 453 bp, Homo sapiens
atcttgtcatatggctaactggctttcagagaattgtgtcttagcaaattaatcatttattaagttagcttttggggaagtaattttcagtaaatttgtataagtaaattttactgcttaaaaccagtcagcctttctaaattcttgttgctagcaatggcaagatcagcaagagggatgatcttacctagagcacactgaggtctgtgagactactaagtccagctgtaagtgggcacaaggccaaaaaatgggcaattttatttaagtcaggaaaactctattcatttgttttatgtagatggtagagaaaagtttctcatgtccatcttccattcttaaagatggattacttagaggaaaatgtaggtttaaaatgtggaaaatgattgtaataatataacctagaaaaaaagatgtataaaaaagtgcattttcatttcttctttctag

SEQ ID NO: 5 - DNA, 471 bp, Artificial Sequence
  Construct
gtaagaaggaatacgcgtatcttgtcatatggctaactggctttcagagaattgtgtcttagcaaattaatcatttattaagttagcttttggggaagtaattttcagtaaatttgtataagtaaattttactgcttaaaaccagtcagcctttctaaattcttgttgctagcaatggcaagatcagcaagagggatgatcttacctagagcacactgaggtctgtgagactactaagtccagctgtaagtgggcacaaggccaaaaaatgggcaattttatttaagtcaggaaaactctattcatttgttttatgtagatggtagagaaaagtttctcatgtccatcttccattcttaaagatggattacttagaggaaaatgtaggtttaaaatgtggaaaatgattgtaataatataacctagaaaaaaagatgtataaaaaagtgcattttcatttcttctttctag

SEQ ID NO: 6 - DNA, 24 bp, Homo sapiens
gttcacatttattgccaaaagctt

SEQ ID NO: 7 - DNA, 10623 bp, Artificial Sequence
  Construct
ctcgagtcagtgtctggcagtgaatgagctacaaattgttttcatattgcttacctgaaggccagtgcttgtttagctgctgaagaaaaatagaaaccttatggcatttagaacatagtttattctttaagtgcagaagtgtgtgacttaacccttgactggcatggtcttagctcctgtttacaatttggtatcttactgccacaaagagtctgttctatcagtcttacattctctattttcacatcaatgctggccagttgtgtctaaacactgcaaaagggagggtatataacaagatatgtctgacttcctgagccatcatggctgggaactcagtttttaagatttatctagggtccatttggccaagattggggtggggagggtctgttcagtcagttgagggctttaagatttatttttagtttacaggaggaaccatgttcagactaccaaatagtttccaatgctgagcagggctagggccacagacacacactcctgtttccatagaaattcctcaggcaggcctcatgtacttagccaggaccctgctcttgcccccaaaggaatttatccttttaaaagtataatattcctcttatctgactgttctactgcacagtggggtaaccacagttaacaatatcatatactatatttcaaaatagctattaatagaagagagaattttgaatattctcaccataaagaaatgacaaatgtttgaagtgattgatatgctaattaccctgatttgatcattacataatgtaaacatgtatagaaacatcacttaaatataaacatgcacaattactagatgtcagttaaaaacaaaataaaacttagaaaagtataatgtgatgatactggttcagggatctgatctttaatccgaaggaaaagaggagacatgaaagaactgaggggagggcttgttattcctcttcctcccctttctctattaccacccttgcattaacatgcccctggttatcttgctatgttttttatatttattatgaatgcagaacacagcaaaaagtaaatagcaattatatcagttctaattctgcttcaagtaatagaacaactcaaagcttattaaattaaagggagaatgtattggttcacgtaattgaaaagtcttgaaatggggctagcaggctgtactcactccaaggctcaaagtatatcatcaagattgggtgagtctctcaatctctcagctctgcttttccctctgttgactacagttttgacatgtagtcatgagatggctgcagcagcttggcctacattcttccctgttcaaatctcaaggtgcaggggccaaggggttggggggagaggtggaggaggaatgctatgtctttattggtagttcacacaatagtcctgagattcactctgccttagacatcccaggtcacatgcttacccatccctgtccactaacatccaagcaacatcttcaccctcataagtcaaagataaactcaccccctcaccctgaccacaaggagtaagaatgggcaatggttggattcttaaatgaaaatcagggactgttgccagaaaaaaaaaaaagtggaatgtatacaatggaggaaaacaataaatgtactagagcaatttttgctactttttttgtagtatttgtattcaacattcaatattctcctgaagctactcttttcctaaaacagatgaaaaccttctccccgatcttctagagatgatcacactgaaccctaaaggacatttaaaaacctccataatcacacagagagggactcaattaaaaaaaattcttggaaaagaaaaaggaaaagatgtatgtttcttgctcttctctcttgatggaaacaaacacacatgcaaaaattcaattgcaataagtgtctcatattgagtttaccaattctgtaagacttcatagttatataaaactaataattaccatttactgaataccttgctatcatgctaggcaaagtgtcagaccattgacatataattcatttaatatactgaagaactatcaaaatgaagtattattatctttactctatagatgaggaaacagacccagaaaggttaagaaatttgctcaaagtcatagagctggcaactggaggagccagtagtcaaaggcaagtctgtgaggtgtgtgagattgtgctctcaacactacaccatggattttgtgatcttggctctaattagggggccaatatggtggacatggtgttatttgaatttaaccatttttcatatctcatatgaaaagttcctagaattaaaaatttcatgaagaaaacaaaaaatagctgaggatttctagatgcatactgtgaaaagaaatgcaaatattaatagtaaccatgttctatgaattagtttaagcatatacatgaaatgctgttagagtacacgttgcaagagtaactaactgatggcctgattagaaaaatgatagaatgaggtaaagaaagaggtagattctgggaaatatggattaaaatgatgaaacaaaaaatgagaaaaaaactaccttaccttctcaagtattgccatagaagagcagaagtcatcctgagcaaagctaaaggcaaaaaacctaggctccagactgatacactggcttgataatccacactgtgagatgtcaccaaaagttgtctgatccagctgtggggacttggctccaagttgtctggatatcctaaaataatataaatcccttgaaacaagtgctctaagaaatggaggaagccatactttagggaatgctgataaaatgagtggaaatgttggtggtgtgagtccacctctacctcaacaaggtcaccactgatgtgagaatccatgaggttaacaaaaacgagcatcattagtctcataaattaacacttgaaggactttggagccttgggtctcagtgattcttctggaaatatggtagtttagggtctttaataatttttttaacttttgttatgagcagaaattgtaaataacatgtaaatacaactccagagtggttctgatgttcaccttctgaactcatctagttctcaaactactagattatgcaacccttgtgctcctacagctaaaccacatatactattctgagccctcccctaactacacctgggtaagaggagtgtggtgtagtctggtgttgtgctataccaatagtcatggagatatcaatcaaacatctgcctaatctacaaggtgtaggtggtgaattatgaagaatgagtaaccaggagacttacgatgagagaataaagtctaagttgataaaaagttagtgctgagtagatagaggggtcttcaggaattggaagcttggtgagggtccccatcatccagaataagtactggtcttcagcaggaagaatttaagaatagaagttacctaaagctcatgaattagtatcagttcaattatgctggtaatctgattttgatctttctcagttatgataaaagtctctgcctggtcatgatataaaccttctttgttctttagtcttccctacttacaagattaaagtcctggcctaaaatatgagccctaaggtcctgctacctagtgacatgatattgttgataccaactctccattacccactgcctactgaaatctcctattcctgccttctccatcattgagttctgtccacctgtcagagtctacttcattgggttgccctccctaacctgcccaacaccaactgcttgccaggccgctaacctattccctcttgttctgcaccttctctcatagtccacttccaatgcatcaccaagatctgcctctaaaatgtatcttctcttcaatcccactgccaatgccttagtttgagattttactatctcttccttcttttatataaattgcctgtatccagctcaatccgccatccatacttccagtagttacatttactgaatgaaaatctggtcctgtcacttttccacttaaaaacccttcagttgccacccaatataaataatatatacatcacttagaggcttccaaggcttccaagccctttccatatgacctttcaggcctgcctctccctgatctttcccatacaatccaataagtgcttttattgttctttaatgttctttaatgtcctttaatgttcagctcaaatattacctccccttgacctctctaaaagaaaacagcttgcttcctcaagtaaagctatttactaatacctaaggtattcctaaaggtatttacaaatacctttattataacacatatcatactgtttatatgtatgctttcttcattagattataaactcttggaggacaaggagcatgccttcttcatttacagttctgtgacaagcatggtgactggcaagtagtcactggcaagaaatgtttcttgaatgaataaatgatccctaaatactgtgacctatctcttagtctgaatttccctcagttacctgcagaaattttccctctggaatatattcttgtttatctatctgtctacctgtctgtctgtctgtctattctatctatctataatctccctatatacaaaggaaacaggtgagaaaggagagtagaagcttatttcaagttccagtccctcctgatctacattctcctgtaattattagcctatgttaccatgtctgaacagaaaatattggtgatgcccttgatcatgaatagctatcatgtctctgttctggcttgctcctggatttttttttttccagtttcatgatgctgcatcactctgtcaccagtcctcactgttcgtttctatggaacatgaaatggtgaatggtccatcctttactgctgatactagtcattgctgaaacagccaccctaaagatctcacagtctctctgtttataaacatttaagagtcaagtcatgaaggctttctcccttaactacatgggatgatcagttgttttatctgttattttttctgttattacttttgtccttaattgttagagaaaactttcatataacacagttactacataaatgccccctcccttcacatcttagaatgcctctggccatctctgtagttgtacttgagaggtttaataaagtaccataagtttgggaaaattagctttattgatataagcacttcctggagaatatttcttcactatacaaaaagctcatatttgatcattgttttctctatatctgtctatctctctcacatacattctgagtgcctgtgtgtgtgtgtgtgtccccagtgacttctgcatctgtagtagctggaataacatgtgtgtcacatgtacccccaccacacacacacacacacacacacctctgccacgggcttcgttcaattctattctcaatttccaaccttgtcagccatcttgatttcctcttctccaaacctcaaagaaactgttaatgagtacccgagaatgaaaatgttgggcatatggaaaaactgaaagacaatccatattgccataaaatggcctgcttttatctggaaaagccttattatcactcacactcattccttctacatcctttacctccttctctttgtctttctgtctgtctctcattctgtctctatcacacacacacacacaacacacacagagagagagagagggagagagagagagagagagagagaaagagagactggctataaagaaagcagcatctgagggcatcaatggtaattcgaacattttgtttgcttgggatcaagattcctttccatcaccattgtcctggcaaatataagctgcaagtggaagtgttttagccagatactgctccaccactctatggctaactaagcaggaatatcagcttcaactatgccatggaattcaagagaatattcaacctggaaaaattctaaccccaaacagcacctcccaaaagatgactacagctcctgtaggaaatcatttaaccacaaattccaactccccttcactcctacagcctcagtcacacatctcaaagggctgatccttgaattgtgacaacctgacccacatcagcagcccagaggccagatggcaacacaatgcctttccagcctactgggtaggaaaagggagggacaagcaattgaatgattataattgaaatcctgtaatttattatttgtcaataactcctgccttgggggagttcccttcactccttagcaaatgctggcagccgcaatatgtgaccacagacacctaaaacaccactgaaagcatttcattatgtggcaacaatgatatggagaataagatctctctggagataagaagacatgccacatctcagagttcactattcaacagcaaagaatttgaatagggggaaaaattccttagacttagggggaaaaattcctgagattctgagggtaaaaagctagcatgcaagtgggatcagccagactggcaggaagtggggcatgaaaacccaagaactatcctcctgtttgcagtataatgttacccctgcagttattaaactgcacagatcaaataactatttgaactgagctgagctgagtcaagcaaagaaatgtacttcaatcatagaatagtagctgtcaggttgaaaagggcatctatcagacatctacccaatgttcctctacctaactcccatcctaattatccctgtctcatgttcatctccctgtaattgatgcaacctgatgcaacataaagaggatttggagtgaaaagccctgagttggaggcctgggccttgactttttaatttactagccataccacattggtccttctagtcttcagctacaatgtgagcagattaaactagttgatgccaagattccatccagttcaaaattctctaggcaataatagggaactcactatctccaaaaataattatttgcatgtttggacaattctattaaaaagtcgtcacttacataaagccaaaacatggtttcttatagtttctacatatttatctgagttctacctgccttgagattatatagtccaagtataatccctcttctatataacagccactcatatagccaaagacagatattgcatctcccatccatctatccatccatccatccatccatccatcccactgagttctgtttgccactcatctagacaatcattctatctactttactcagaacacataccaatttgtccatatccttactgaaaaatggtcatcagaactgaacaaagagctacagaaatagtttgataaatgctgagccaagtacctcaccacctattttgttccagctaatgtgtttattttaatgcagcctaatatcacattatctttttggctatggcatcactctgccactgccacaatattgctttttttactcatgtcttctaccctatccttgtaaagtgtaatttggaactggagtcagaattttactttttcaataatattcatttatcagatttgagccattggtgcaacatgataaaatcttcttgaatcctcagtctatcctaaaaatatttgccatctatccaaattttcacctaagttattgataaaatacaatgagcacaacatggtcaggaaaaaagtcctagagcagaccactaaacaataccctccatattgacataattctgcaagatttggggtcttttggccccaaagaaggtcatatccaagattgcctcattcttgacactctctcaatgctaccttcttcactcactcatagtccatggtctagtctttctgctgatcctgcaatgatttccacagttaagttcacccagacataggtactttgagagaaatcactgtttaaaacattaaaaatataaataaggttaacaaaaaaaggataaaagttcacacccagaacaatatgccatggtggaaataatgtgggagttggagtctgaataacctcagattaaatcctagttctgtaacttacttgcttggtgaccttgggtgccttatcagtatcagttccctcatctctaaaatagaggaataataatgacctcaaagggttgtcctgatgattaaaattaatacacgcaaaatcttagcacattcctagcacataatgggtatacaatatagattactatcattattacatcatggtagagacaaaaatgcaataactactggcataaaaaaattagacccaagaagaaaaaaggagaaaatgaatatctgtgttctacagatgttaaccaatactccacaaaagaaagaatcaatggtctaataaggttaagaacgaggacattaaacacagttttaaaaactcgggcatatttttatctctttgactctgaacaagattggcacacccttgcatcccttatttgatcatgtcccaggcattgctgtagggcccaaggacacaaaggatgactcagacccagtcaatgctagggtgagaagaaacacaggattcaaggcaccagggggcagggggcacacatataaataagtaattaatataaaatatcaccagtgctattgagaggtaaatacagagtatgtgagggaacactgataaatctggagatgtcaggtaaggctttatgcaggaggtggtaattttgaagaatcttaaaagataaagagaagttaaggatgagggtctctcaagtcaaggtagtggaggatagcacgcaaagattttgaacagtagtggaccaatatgggctcgtccaagaaatattgctcattctagagtgacttttccatgagatacaggtaataagagggacaattgaaaagatacagattcatgcctattgtaaaggggcttgtacgtcaggtaaaaaagtctgaaattctgcaggcaagaagaaaccagcacagcgaatgacataagatggtaagccagcttaatagtagagagagaggctagttacgacacttttttttctttaccaatacccaattgaaagatggtcaggtccagaactaaaattatagcactggaggtagagaataggcctacataacaaaatctaagcctgagtataattgatacgatgtgacatgagcatgaggggttaaggaaaaggagaaatgaaaatgactttgtctagcttgagacacccagtgtgtgggggcatcattaccaatatatgggcttctggaggatagtacattttggtaggaggcacaatgtaagttcagttttcaacactatggattgagataaccatgggacatccatatggagatgcaatctgctcaacataacagtgtgtatatcataagacagaccagggataggagtcccctgtatatttatggcaataaaagaaaattcatggtttaagaaggaagagtatgtggaacatgtctctgatgccacgatgtaataaccttgaaaacaaagtaaaattacactaatgagtcttgcctaattaaactcatgctcctagtgatcaccacttctagttcaattgttcacattcttgctctgctttgaaaaattaaaattaaatttgcctatcctctactgaccataatttctagaagacggcattcatctcatggcaagttcttcagtacccaaagatggaatacatagattaaaaaagaacatatatgtagatgcttgtgatgttttcctatcataaattgaatttcaagttcttataaacgtattaatatgtcctactcttctagagacaaggatcaggaagtgtatttatcaatagatatttaccaagcacctgtcaagccaaagtggggttacagaaaagtaggtatgggccctgcacacaaacaacctgtattagccaaagggacccttccataaaatttccaatatgtaaacccaaatttggaacttgctgaaacaagtacagatgagtacgtgaggaagctgggaagtaaacacaggttgctggagaaatagaggtggagatatgggtggatctaggtttggttaggaatgaatcagaccatcccacagagggtggctcctccctgcatggggcctgctataaaagggccattatctcagccttcagtacccagcaggctccttcaggctacattctatttgctcttttggtgaacaaggtaagaaggaatacgcgtatcttgtcatatggctaactggctttcagagaattgtgtcttagcaaattaatcatttattaagttagcttttggggaagtaattttcagtaaatttgtataagtaaattttactgcttaaaaccagtcagcctttctaaattcttgttgctagcaatggcaagatcagcaagagggatgatcttacctagagcacactgaggtctgtgagactactaagtccagctgtaagtgggcacaaggccaaaaaatgggcaattttatttaagtcaggaaaactctattcatttgttttatgtagatggtagagaaaagtttctcatgtccatcttccattcttaaagatggattacttagaggaaaatgtaggtttaaaatgtggaaaatgattgtaataatataacctagaaaaaaagatgtataaaaaagtgcattttcatttcttctttctaggttcacatttattgccaaaagctt

SEQ ID NO: 8 - DNA, 76 bp, Homo sapiens
ggtaagcaatatgaaaacaatttgtagctcattcactgccagacactgactcgagacaacttatatcgtatggggc

SEQ ID NO: 9 - DNA, 76 bp, Homo sapiens; identical to SEQ ID NO: 8

SEQ ID NO: 10 - DNA, 76 bp, Homo sapiens
tccttcaggctacattctatttgctcttttggtgaacaaggtaagaaggaatacgcgttacgccccgccctgccac

SEQ ID NO: 11 - DNA, 76 bp, Homo sapiens
gtggcagggcggggcgtaacgcgtattccttcttaccttgttcaccaaaagagcaaatagaatgtagcctgaagga

SEQ ID NO: 12 - DNA, 10146 bp, Artificial Sequence
  Cloned sequence
ctcgagtcagtgtctggcagtgaatgagctacaaattgttttcatattgcttacctgaaggccagtgcttgtttagctgctgaagaaaaatagaaaccttatggcatttagaacatagtttattctttaagtgcagaagtgtgtgacttaacccttgactggcatggtcttagctcctgtttacaatttggtatcttactgccacaaagagtctgttctatcagtcttacattctctattttcacatcaatgctggccagttgtgtctaaacactgcaaaagggagggtatataacaagatatgtctgacttcctgagccatcatggctgggaactcagtttttaagatttatctagggtccatttggccaagattggggtggggagggtctgttcagtcagttgagggctttaagatttatttttagtttacaggaggaaccatgttcagactaccaaatagtttccaatgctgagcagggctagggccacagacacacactcctgtttccatagaaattcctcaggcaggcctcatgtacttagccaggaccctgctcttgcccccaaaggaatttatccttttaaaagtataatattcctcttatctgactgttctactgcacagtggggtaaccacagttaacaatatcatatactatatttcaaaatagctattaatagaagagagaattttgaatattctcaccataaagaaatgacaaatgtttgaagtgattgatatgctaattaccctgatttgatcattacataatgtaaacatgtatagaaacatcacttaaatataaacatgcacaattactagatgtcagttaaaaacaaaataaaacttagaaaagtataatgtgatgatactggttcagggatctgatctttaatccgaaggaaaagaggagacatgaaagaactgaggggagggcttgttattcctcttcctcccctttctctattaccacccttgcattaacatgcccctggttatcttgctatgttttttatatttattatgaatgcagaacacagcaaaaagtaaatagcaattatatcagttctaattctgcttcaagtaatagaacaactcaaagcttattaaattaaagggagaatgtattggttcacgtaattgaaaagtcttgaaatggggctagcaggctgtactcactccaaggctcaaagtatatcatcaagattgggtgagtctctcaatctctcagctctgcttttccctctgttgactacagttttgacatgtagtcatgagatggctgcagcagcttggcctacattcttccctgttcaaatctcaaggtgcaggggccaaggggttggggggagaggtggaggaggaatgctatgtctttattggtagttcacacaatagtcctgagattcactctgccttagacatcccaggtcacatgcttacccatccctgtccactaacatccaagcaacatcttcaccctcataagtcaaagataaactcaccccctcaccctgaccacaaggagtaagaatgggcaatggttggattcttaaatgaaaatcagggactgttgccagaaaaaaaaaaaagtggaatgtatacaatggaggaaaacaataaatgtactagagcaatttttgctactttttttgtagtatttgtattcaacattcaatattctcctgaagctactcttttcctaaaacagatgaaaaccttctccccgatcttctagagatgatcacactgaaccctaaaggacatttaaaaacctccataatcacacagagagggactcaattaaaaaaaattcttggaaaagaaaaaggaaaagatgtatgtttcttgctcttctctcttgatggaaacaaacacacatgcaaaaattcaattgcaataagtgtctcatattgagtttaccaattctgtaagacttcatagttatataaaactaataattaccatttactgaataccttgctatcatgctaggcaaagtgtcagaccattgacatataattcatttaatatactgaagaactatcaaaatgaagtattattatctttactctatagatgaggaaacagacccagaaaggttaagaaatttgctcaaagtcatagagctggcaactggaggagccagtagtcaaaggcaagtctgtgaggtgtgtgagattgtgctctcaacactacaccatggattttgtgatcttggctctaattagggggccaatatggtggacatggtgttatttgaatttaaccatttttcatatctcatatgaaaagttcctagaattaaaaatttcatgaagaaaacaaaaaatagctgaggatttctagatgcatactgtgaaaagaaatgcaaatattaatagtaaccatgttctatgaattagtttaagcatatacatgaaatgctgttagagtacacgttgcaagagtaactaactgatggcctgattagaaaaatgatagaatgaggtaaagaaagaggtagattctgggaaatatggattaaaatgatgaaacaaaaaatgagaaaaaaactaccttaccttctcaagtattgccatagaagagcagaagtcatcctgagcaaagctaaaggcaaaaaacctaggctccagactgatacactggcttgataatccacactgtgagatgtcaccaaaagttgtctgatccagctgtggggacttggctccaagttgtctggatatcctaaaataatataaatcccttgaaacaagtgctctaagaaatggaggaagccatactttagggaatgctgataaaatgagtggaaatgttggtggtgtgagtccacctctacctcaacaaggtcaccactgatgtgagaatccatgaggttaacaaaaacgagcatcattagtctcataaattaacacttgaaggactttggagccttgggtctcagtgattcttctggaaatatggtagtttagggtctttaataatttttttaacttttgttatgagcagaaattgtaaataacatgtaaatacaactccagagtggttctgatgttcaccttctgaactcatctagttctcaaactactagattatgcaacccttgtgctcctacagctaaaccacatatactattctgagccctcccctaactacacctgggtaagaggagtgtggtgtagtctggtgttgtgctataccaatagtcatggagatatcaatcaaacatctgcctaatctacaaggtgtaggtggtgaattatgaagaatgagtaaccaggagacttacgatgagagaataaagtctaagttgataaaaagttagtgctgagtagatagaggggtcttcaggaattggaagcttggtgagggtccccatcatccagaataagtactggtcttcagcaggaagaatttaagaatagaagttacctaaagctcatgaattagtatcagttcaattatgctggtaatctgattttgatctttctcagttatgataaaagtctctgcctggtcatgatataaaccttctttgttctttagtcttccctacttacaagattaaagtcctggcctaaaatatgagccctaaggtcctgctacctagtgacatgatattgttgataccaactctccattacccactgcctactgaaatctcctattcctgccttctccatcattgagttctgtccacctgtcagagtctacttcattgggttgccctccctaacctgcccaacaccaactgcttgccaggccgctaacctattccctcttgttctgcaccttctctcatagtccacttccaatgcatcaccaagatctgcctctaaaatgtatcttctcttcaatcccactgccaatgccttagtttgagattttactatctcttccttcttttatataaattgcctgtatccagctcaatccgccatccatacttccagtagttacatttactgaatgaaaatctggtcctgtcacttttccacttaaaaacccttcagttgccacccaatataaataatatatacatcacttagaggcttccaaggcttccaagccctttccatatgacctttcaggcctgcctctccctgatctttcccatacaatccaataagtgcttttattgttctttaatgttctttaatgtcctttaatgttcagctcaaatattacctccccttgacctctctaaaagaaaacagcttgcttcctcaagtaaagctatttactaatacctaaggtattcctaaaggtatttacaaatacctttattataacacatatcatactgtttatatgtatgctttcttcattagattataaactcttggaggacaaggagcatgccttcttcatttacagttctgtgacaagcatggtgactggcaagtagtcactggcaagaaatgtttcttgaatgaataaatgatccctaaatactgtgacctatctcttagtctgaatttccctcagttacctgcagaaattttccctctggaatatattcttgtttatctatctgtctacctgtctgtctgtctgtctattctatctatctataatctccctatatacaaaggaaacaggtgagaaaggagagtagaagcttatttcaagttccagtccctcctgatctacattctcctgtaattattagcctatgttaccatgtctgaacagaaaatattggtgatgcccttgatcatgaatagctatcatgtctctgttctggcttgctcctggatttttttttttccagtttcatgatgctgcatcactctgtcaccagtcctcactgttcgtttctatggaacatgaaatggtgaatggtccatcctttactgctgatactagtcattgctgaaacagccaccctaaagatctcacagtctctctgtttataaacatttaagagtcaagtcatgaaggctttctcccttaactacatgggatgatcagttgttttatctgttattttttctgttattacttttgtccttaattgttagagaaaactttcatataacacagttactacataaatgccccctcccttcacatcttagaatgcctctggccatctctgtagttgtacttgagaggtttaataaagtaccataagtttgggaaaattagctttattgatataagcacttcctggagaatatttcttcactatacaaaaagctcatatttgatcattgttttctctatatctgtctatctctctcacatacattctgagtgcctgtgtgtgtgtgtgtgtccccagtgacttctgcatctgtagtagctggaataacatgtgtgtcacatgtacccccaccacacacacacacacacacacacctctgccacgggcttcgttcaattctattctcaatttccaaccttgtcagccatcttgatttcctcttctccaaacctcaaagaaactgttaatgagtacccgagaatgaaaatgttgggcatatggaaaaactgaaagacaatccatattgccataaaatggcctgcttttatctggaaaagccttattatcactcacactcattccttctacatcctttacctccttctctttgtctttctgtctgtctctcattctgtctctatcacacacacacacacaacacacacagagagagagagagggagagagagagagagagagagagaaagagagactggctataaagaaagcagcatctgagggcatcaatggtaattcgaacattttgtttgcttgggatcaagattcctttccatcaccattgtcctggcaaatataagctgcaagtggaagtgttttagccagatactgctccaccactctatggctaactaagcaggaatatcagcttcaactatgccatggaattcaagagaatattcaacctggaaaaattctaaccccaaacagcacctcccaaaagatgactacagctcctgtaggaaatcatttaaccacaaattccaactccccttcactcctacagcctcagtcacacatctcaaagggctgatccttgaattgtgacaacctgacccacatcagcagcccagaggccagatggcaacacaatgcctttccagcctactgggtaggaaaagggagggacaagcaattgaatgattataattgaaatcctgtaatttattatttgtcaataactcctgccttgggggagttcccttcactccttagcaaatgctggcagccgcaatatgtgaccacagacacctaaaacaccactgaaagcatttcattatgtggcaacaatgatatggagaataagatctctctggagataagaagacatgccacatctcagagttcactattcaacagcaaagaatttgaatagggggaaaaattccttagacttagggggaaaaattcctgagattctgagggtaaaaagctagcatgcaagtgggatcagccagactggcaggaagtggggcatgaaaacccaagaactatcctcctgtttgcagtataatgttacccctgcagttattaaactgcacagatcaaataactatttgaactgagctgagctgagtcaagcaaagaaatgtacttcaatcatagaatagtagctgtcaggttgaaaagggcatctatcagacatctacccaatgttcctctacctaactcccatcctaattatccctgtctcatgttcatctccctgtaattgatgcaacctgatgcaacataaagaggatttggagtgaaaagccctgagttggaggcctgggccttgactttttaatttactagccataccacattggtccttctagtcttcagctacaatgtgagcagattaaactagttgatgccaagattccatccagttcaaaattctctaggcaataatagggaactcactatctccaaaaataattatttgcatgtttggacaattctattaaaaagtcgtcacttacataaagccaaaacatggtttcttatagtttctacatatttatctgagttctacctgccttgagattatatagtccaagtataatccctcttctatataacagccactcatatagccaaagacagatattgcatctcccatccatctatccatccatccatccatccatccatcccactgagttctgtttgccactcatctagacaatcattctatctactttactcagaacacataccaatttgtccatatccttactgaaaaatggtcatcagaactgaacaaagagctacagaaatagtttgataaatgctgagccaagtacctcaccacctattttgttccagctaatgtgtttattttaatgcagcctaatatcacattatctttttggctatggcatcactctgccactgccacaatattgctttttttactcatgtcttctaccctatccttgtaaagtgtaatttggaactggagtcagaattttactttttcaataatattcatttatcagatttgagccattggtgcaacatgataaaatcttcttgaatcctcagtctatcctaaaaatatttgccatctatccaaattttcacctaagttattgataaaatacaatgagcacaacatggtcaggaaaaaagtcctagagcagaccactaaacaataccctccatattgacataattctgcaagatttggggtcttttggccccaaagaaggtcatatccaagattgcctcattcttgacactctctcaatgctaccttcttcactcactcatagtccatggtctagtctttctgctgatcctgcaatgatttccacagttaagttcacccagacataggtactttgagagaaatcactgtttaaaacattaaaaatataaataaggttaacaaaaaaaggataaaagttcacacccagaacaatatgccatggtggaaataatgtgggagttggagtctgaataacctcagattaaatcctagttctgtaacttacttgcttggtgaccttgggtgccttatcagtatcagttccctcatctctaaaatagaggaataataatgacctcaaagggttgtcctgatgattaaaattaatacacgcaaaatcttagcacattcctagcacataatgggtatacaatatagattactatcattattacatcatggtagagacaaaaatgcaataactactggcataaaaaaattagacccaagaagaaaaaaggagaaaatgaatatctgtgttctacagatgttaaccaatactccacaaaagaaagaatcaatggtctaataaggttaagaacgaggacattaaacacagttttaaaaactcgggcatatttttatctctttgactctgaacaagattggcacacccttgcatcccttatttgatcatgtcccaggcattgctgtagggcccaaggacacaaaggatgactcagacccagtcaatgctagggtgagaagaaacacaggattcaaggcaccagggggcagggggcacacatataaataagtaattaatataaaatatcaccagtgctattgagaggtaaatacagagtatgtgagggaacactgataaatctggagatgtcaggtaaggctttatgcaggaggtggtaattttgaagaatcttaaaagataaagagaagttaaggatgagggtctctcaagtcaaggtagtggaggatagcacgcaaagattttgaacagtagtggaccaatatgggctcgtccaagaaatattgctcattctagagtgacttttccatgagatacaggtaataagagggacaattgaaaagatacagattcatgcctattgtaaaggggcttgtacgtcaggtaaaaaagtctgaaattctgcaggcaagaagaaaccagcacagcgaatgacataagatggtaagccagcttaatagtagagagagaggctagttacgacacttttttttctttaccaatacccaattgaaagatggtcaggtccagaactaaaattatagcactggaggtagagaataggcctacataacaaaatctaagcctgagtataattgatacgatgtgacatgagcatgaggggttaaggaaaaggagaaatgaaaatgactttgtctagcttgagacacccagtgtgtgggggcatcattaccaatatatgggcttctggaggatagtacattttggtaggaggcacaatgtaagttcagttttcaacactatggattgagataaccatgggacatccatatggagatgcaatctgctcaacataacagtgtgtatatcataagacagaccagggataggagtcccctgtatatttatggcaataaaagaaaattcatggtttaagaaggaagagtatgtggaacatgtctctgatgccacgatgtaataaccttgaaaacaaagtaaaattacactaatgagtcttgcctaattaaactcatgctcctagtgatcaccacttctagttcaattgttcacattcttgctctgctttgaaaaattaaaattaaatttgcctatcctctactgaccataatttctagaagacggcattcatctcatggcaagttcttcagtacccaaagatggaatacatagattaaaaaagaacatatatgtagatgcttgtgatgttttcctatcataaattgaatttcaagttcttataaacgtattaatatgtcctactcttctagagacaaggatcaggaagtgtatttatcaatagatatttaccaagcacctgtcaagccaaagtggggttacagaaaagtaggtatgggccctgcacacaaacaacctgtattagccaaagggacccttccataaaatttccaatatgtaaacccaaatttggaacttgctgaaacaagtacagatgagtacgtgaggaagctgggaagtaaacacaggttgctggagaaatagaggtggagatatgggtggatctaggtttggttaggaatgaatcagaccatcccacagagggtggctcctccctgcatggggcctgctataaaagggccattatctcagccttcagtacccagcaggctccttcaggctacattctatttgctcttttggtgaacaaggtaagaaggaatacgcgt

SEQ ID NO: 13 - DNA, 37 bp, Artificial Sequence
  Primer
aggtaagtcacgcgtatcttgtcatatggctaactgg

SEQ ID NO: 14 - DNA, 27 bp, Artificial Sequence
  Primer
tcaagcttttggcaataaatgtgaacc

SEQ ID NO: 15 - DNA, 483 bp, Artificial Sequence
  Cloned sequence
acgcgtatcttgtcatatggctaactggctttcagagaattgtgtcttagcaaattaatcatttattaagttagcttttggggaagtaattttcagtaaatttgtataagtaaattttactgcttaaaaccagtcagcctttctaaattcttgttgctagcaatggcaagatcagcaagagggatgatcttacctagagcacactgaggtctgtgagactactaagtccagctgtaagtgggcacaaggccaaaaaatgggcaattttatttaagtcaggaaaactctattcatttgttttatgtagatggtagagaaaagtttctcatgtccatcttccattcttaaagatggattacttagaggaaaatgtaggtttaaaatgtggaaaatgattgtaataatataacctagaaaaaaagatgtataaaaaagtgcattttcatttcttctttctaggttcacatttattgccaaaagctt

SEQ ID NO: 16 - DNA, 10623 bp, Homo sapiens; identical to SEQ ID NO: 7

SEQ ID NO: 17 - DNA, 15 bp, Homo sapiens
cttttggtgaacaag

SEQ ID NO: 18 - DNA, 21 bp, Homo sapiens
gttcacatttattgccaaaag

(19) United States
(12) Patent Application Publication
(10) Pub. No.: US 2015/0342164 A1
(43) Pub. Date: Dec. 3, 2015

(54) ANIMAL MODEL WITH A FILAGGRIN GENE PROMOTER

(71) Applicant: UNIVERSITY OF DUNDEE, Dundee (GB)

(72) Inventors: W.H. Irwin McLean, Dundee (GB); Vikas Hedge, Dundee (GB); Deena M. Leslie Pedrioli, Dundee (GB); Sitheswaran Nainamalai, Lausanne (CH); Paul A. Campbell, Dundee (GB); Frances J.D. Smith, Dundee (GB)

(21) Appl. No.: 14/654,325

(22) PCT Filed: Dec. 19, 2013
(86) PCT No.: PCT/GB2013/053367

§ 371 Date: Jun. 19, 2015

(30) Foreign Application Priority Data
Dec. 19, 2012 (GB) 1222881.3

(51) Int. Cl. A01K 67/027; C07K 14/47; G01N 33/573; C12N 15/85; A61K 49/00; C12Q 1/68

(52) U.S. Cl. CPC A01K 67/0278; A61K 49/0008; C12Q 1/6897; G01N 33/573; C12N 15/8509; C07K 14/47; A01K 2227/105; A01K 2267/0368; G01N 2333/90241; G01N 2500/10; G01N 2500/02; C12Q 2600/136; C12Q 2600/158; C12N 2830/008

(57) ABSTRACT

The present invention is based upon the generation of a nucleic acid construct which may be used to generate transgenic non-human animals. The construct exploits a filaggrin based promoter region which directs the expression of a reporter sequence operatively linked thereto. The present invention provides a nucleic acid, said nucleic acid encoding a filaggrin promoter element and a nucleic acid sequence operatively linked thereto.

### FIELD OF THE INVENTION

The present invention provides nucleic acid constructs and uses of the same for generating transgenic, non-human animals. The invention further relates to the use of such animals in method for testing agents for potential utility in the treatment of filaggrin based disorders.

### BACKGROUND OF THE INVENTION

Following the discovery of the major role of the filaggrin gene in dry skin and atopic eczema, filaggrin has emerged as a potential new drug target for treatment of these diseases. Specifically, it is desirable to develop new drugs that can up-regulate the expression of the filaggrin gene in the skin. There are few tools, if any, available to allow the development of such compounds and, in particular, there is no in vivo model to validate filaggrin up-regulation or study filaggrin expression in real time.

It is amongst the objects of the present invention to obviate and/or instigate one or more of the aforementioned disadvantages

### SUMMARY OF THE INVENTION

The present invention is based upon the generation of a nucleic acid construct which may be used to generate transgenic non-human animals. The construct exploits a filaggrin based promoter region which directs the expression of a reporter sequence operatively linked thereto.

It should be noted that throughout this specification the term comprising is used to denote that embodiments of the invention “comprise” the noted features and as such, may also include other features. However, in the context of this invention, the term “comprising” encompasses embodiments in which the invention “consists essentially of” the relevant features or “consists of” the relevant features. For example, while this invention provides isolated nucleic acid sequences which comprise certain sequences, the invention further relates to isolated sequences which “consist essentially” or “consist” of the same sequences.

In a first aspect, the present invention provides a nucleic acid, said nucleic acid encoding a filaggrin promoter element and a nucleic acid sequence operatively linked thereto.

The nucleic acid sequence operatively linked thereto may not be a sequence encoding the complete, functional, wild-type and/or native human filaggrin protein. In other words, the operatively linked nucleic acid sequence may not be the complete human filaggrin (FLG) gene. Moreover, the nucleic acid of this invention may be an isolated nucleic acid. Furthermore, the nucleic acid may be artificially constructed using molecule/recombinant techniques such as, for example, cloning, PCR, ligation and the like. Hereinafter, the nucleic acid sequence of the first aspect of this invention shall be referred to as a nucleic acid “construct”.

Within this specification, reference is made to sequences which “exhibit a degree of identity or homology” to, for example, a reference sequence. As used herein, the term “degree of homology” or “degree of identity” may encompass nucleic acid and/or amino acid sequences which exhibit at least about 30%, 40%, 50%, 60%, 65%, 70%, 75%, 80%, 85%, 90%, 91%, 92%, 93%, 94%, 95%, 96%, 97%, 98% or 99% homology or identity to a reference nucleic acid or amino acid sequence. In the context of this specification, the reference nucleic acid sequence may be a coding and/or non-coding sequence of the human filaggrin gene. For example, the reference sequence may be a coding/non-coding sequence of a (wild-type or native) human filaggrin promoter/gene sequence.

The degree of (or percentage) “homology” between two or more (amino acid or nucleic acid) sequences may be calculated by aligning the sequences and determining the number of aligned residues which are identical and adding this to the number of residues which are not identical but which differ by redundant nucleotide substitutions—the redundant nucleotide substitution having no effect upon the amino acid encoded by a particular codon, or conservative amino acid substitutions. The combined total is then divided by the total number of residues compared and the resulting figure is multiplied by 100—this yields the percentage homology between aligned sequences.

A degree of (or percentage) “identity” between two or more (amino acid or nucleic acid) sequences may also be determined by aligning the sequences and ascertaining the number of exact residue matches between the aligned sequences and dividing this number by the number of total residues compared—multiplying the resultant figure by 100 would yield the percentage identity between the sequences.

The constructs provided by this invention comprise a filaggrin promoter element. A filaggrin promoter element suitable for use may encode a sequence which is capable of directing the expression of a gene or genes–particularly a gene or genes which are located downstream of the promoter element and/or which are operatively linked thereto. Promoter sequences may otherwise be referred to as encoding “transcription regulators”. As such, the filaggrin promoter element of the constructs of this invention may encode a transcription regulator capable of directing the expression of one more sequences operatively linked thereto. Suitable promoter elements may be capable of directing the expression of the human filaggrin gene. Accordingly, the constructs of this invention may comprise at least one human filaggrin gene transcription regulator sequence/element.

The filaggrin promoter element of the constructs of this invention may comprise a sequence which exhibits a degree of identity or homology to a filaggrin promoter sequence, for example the human filaggrin promoter sequence. It should be understood that a filaggrin promoter sequence may be a sequence of the filaggrin promoter which regulates the expression of the filaggrin gene. The promoter element may comprise a sequence which exhibits a degree of identity or homology to a wild type (or native) human filaggrin promoter sequence, the degree of identity/homology being determined by comparison between the sequence of the promoter element of the construct and the sequence of a reference human filaggrin gene promoter sequence.

The promoter element of the constructs described herein may be encoded by a sequence exhibiting a degree of identity and/or homology to a complete (or substantially complete) human filaggrin promoter sequence or to a fragment or portion thereof. A fragment or portion of a human filaggrin promoter sequence may comprise from about x to about n−1 nucleotides (and every number therebetween), where “x” is a nucleic acid fragment comprising from about 10 to about 5000 nucleotide bases (for example, about 10, 20, 30, 40, 50, 100, 500, 1000, 1500, 2000, 2500, 3000, 3500, 4000, 4500, 5000, 10,000 nucleotide bases) and “n” is the total number of nucleotide bases of a reference human filaggrin promoter sequence. Any fragment or portion of a promoter sequence (for example a human filaggrin promoter sequence) may be functionally active. That is to say, fragments or portions of promoter sequences as described herein (including the human filaggrin promoter sequence) may act as transcriptional regulators and be capable of controlling the expression of sequences operatively linked thereto. Accordingly, promoter elements of this invention exhibiting a degree of identity and/or homology to a fragment or portion of a reference sequence, for example a human filaggrin promoter sequence, may be also be functional transcription regulators. Functional fragments of this type will be referred to hereinafter as “promoter fragments”.

In view of the above, the present invention provides a nucleic acid, said nucleic acid encoding a filaggrin promoter element and a nucleic acid sequence operatively linked thereto, wherein the filaggrin promoter element comprises a sequence exhibiting a degree of identity or homology to a wild type or native filaggrin promoter sequence.

An exemplary human filaggrin promoter sequence is provided below as SEQ ID NO: 1.

SEQ ID NO: 1
ctcgagtcagtgtctggcagtgaatgagctacaaattgttttcatattgc
ttacctgaaggccagtgcttgtttagctgctgaagaaaaatagaaacctt
atggcatttagaacatagtttattctttaagtgcagaagtgtgtgactta
acccttgactggcatggtcttagctcctgtttacaatttggtatcttact
gccacaaagagtctgttctatcagtcttacattctctattttcacatcaa
tgctggccagttgtgtctaaacactgcaaaagggagggtatataacaaga
tatgtctgacttcctgagccatcatggctgggaactcagtttttaagatt
tatctagggtccatttggccaagattggggtggggagggtctgttcagtc
agttgagggctttaagatttatttttagtttacaggaggaaccatgttca
gactaccaaatagtttccaatgctgagcagggctagggccacagacacac
actcctgtttccatagaaattcctcaggcaggcctcatgtacttagccag
gaccctgctcttgcccccaaaggaatttatccttttaaaagtataatatt
cctcttatctgactgttctactgcacagtggggtaaccacagttaacaat
atcatatactatatttcaaaatagctattaatagaagagagaattttgaa
tattctcaccataaagaaatgacaaatgtttgaagtgattgatatgctaa
ttaccctgatttgatcattacataatgtaaacatgtatagaaacatcact
taaatataaacatgcacaattactagatgtcagttaaaaacaaaataaaa
cttagaaaagtataatgtgatgatactggttcagggatctgatctttaat
ccgaaggaaaagaggagacatgaaagaactgaggggagggcttgttattc
ctcttcctcccctttctctattaccacccttgcattaacatgcccctggt
tatcttgctatgttttttatatttattatgaatgcagaacacagcaaaaa
gtaaatagcaattatatcagttctaattctgcttcaagtaatagaacaac
tcaaagcttattaaattaaagggagaatgtattggttcacgtaattgaaa
agtcttgaaatggggctagcaggctgtactcactccaaggctcaaagtat
atcatcaagattgggtgagtctctcaatctctcagctctgcttttccctc
tgttgactacagttttgacatgtagtcatgagatggctgcagcagcttgg
cctacattcttccctgttcaaatctcaaggtgcaggggccaaggggttgg
ggggagaggtggaggaggaatgctatgtctttattggtagttcacacaat
agtcctgagattcactctgccttagacatcccaggtcacatgcttaccca
tccctgtccactaacatccaagcaacatcttcaccctcataagtcaaaga
taaactcaccccctcaccctgaccacaaggagtaagaatgggcaatggtt
ggattcttaaatgaaaatcagggactgttgccagaaaaaaaaaaaagtgg
aatgtatacaatggaggaaaacaataaatgtactagagcaatttttgcta
ctttttttgtagtatttgtattcaacattcaatattctcctgaagctact
cttttcctaaaacagatgaaaaccttctccccgatcttctagagatgatc
acactgaaccctaaaggacatttaaaaacctccataatcacacagagagg
gactcaattaaaaaaaattcttggaaaagaaaaaggaaaagatgtatgtt
tcttgctcttctctcttgatggaaacaaacacacatgcaaaaattcaatt
gcaataagtgtctcatattgagtttaccaattctgtaagacttcatagtt
atataaaactaataattaccatttactgaataccttgctatcatgctagg
caaagtgtcagaccattgacatataattcatttaatatactgaagaacta
tcaaaatgaagtattattatctttactctatagatgaggaaacagaccca
gaaaggttaagaaatttgctcaaagtcatagagctggcaactggaggagc
cagtagtcaaaggcaagtctgtgaggtgtgtgagattgtgctctcaacac
tacaccatggattttgtgatcttggctctaattagggggccaatatggtg
gacatggtgttatttgaatttaaccatttttcatatctcatatgaaaagt
tcctagaattaaaaatttcatgaagaaaacaaaaaatagctgaggatttc
tagatgcatactgtgaaaagaaatgcaaatattaatagtaaccatgttct
atgaattagtttaagcatatacatgaaatgctgttagagtacacgttgca
agagtaactaactgatggcctgattagaaaaatgatagaatgaggtaaag
aaagaggtagattctgggaaatatggattaaaatgatgaaacaaaaaatg
agaaaaaaactaccttaccttctcaagtattgccatagaagagcagaagt
catcctgagcaaagctaaaggcaaaaaacctaggctccagactgatacac
tggcttgataatccacactgtgagatgtcaccaaaagttgtctgatccag
ctgtggggacttggctccaagttgtctggatatcctaaaataatataaat
cccttgaaacaagtgctctaagaaatggaggaagccatactttagggaat
gctgataaaatgagtggaaatgttggtggtgtgagtccacctctacctca
acaaggtcaccactgatgtgagaatccatgaggttaacaaaaacgagcat
cattagtctcataaattaacacttgaaggactttggagccttgggtctca
gtgattcttctggaaatatggtagtttagggtctttaataatttttttaa
cttttgttatgagcagaaattgtaaataacatgtaaatacaactccagag
tggttctgatgttcaccttctgaactcatctagttctcaaactactagat
tatgcaacccttgtgctcctacagctaaaccacatatactattctgagcc
ctcccctaactacacctgggtaagaggagtgtggtgtagtctggtgttgt
gctataccaatagtcatggagatatcaatcaaacatctgcctaatctaca
aggtgtaggtggtgaattatgaagaatgagtaaccaggagacttacgatg
agagaataaagtctaagttgataaaaagttagtgctgagtagatagaggg
gtcttcaggaattggaagcttggtgagggtccccatcatccagaataagt
actggtcttcagcaggaagaatttaagaatagaagttacctaaagctcat
gaattagtatcagttcaattatgctggtaatctgattttgatctttctca
gttatgataaaagtctctgcctggtcatgatataaaccttctttgttctt
tagtcttccctacttacaagattaaagtcctggcctaaaatatgagccct
aaggtcctgctacctagtgacatgatattgttgataccaactctccatta
cccactgcctactgaaatctcctattcctgccttctccatcattgagttc
tgtccacctgtcagagtctacttcattgggttgccctccctaacctgccc
aacaccaactgcttgccaggccgctaacctattccctcttgttctgcacc
ttctctcatagtccacttccaatgcatcaccaagatctgcctctaaaatg
tatcttctcttcaatcccactgccaatgccttagtttgagattttactat
ctcttccttcttttatataaattgcctgtatccagctcaatccgccatcc
atacttccagtagttacatttactgaatgaaaatctggtcctgtcacttt
tccacttaaaaacccttcagttgccacccaatataaataatatatacatc
acttagaggcttccaaggcttccaagccctttccatatgacctttcaggc
ctgcctctccctgatctttcccatacaatccaataagtgcttttattgtt
ctttaatgttctttaatgtcctttaatgttcagctcaaatattacctccc
cttgacctctctaaaagaaaacagcttgcttcctcaagtaaagctattta
ctaatacctaaggtattcctaaaggtatttacaaatacctttattataac
acatatcatactgtttatatgtatgctttcttcattagattataaactct
tggaggacaaggagcatgccttcttcatttacagttctgtgacaagcatg
gtgactggcaagtagtcactggcaagaaatgtttcttgaatgaataaatg
atccctaaatactgtgacctatctcttagtctgaatttccctcagttacc
tgcagaaattttccctctggaatatattcttgtttatctatctgtctacc
tgtctgtctgtctgtctattctatctatctataatctccctatatacaaa
ggaaacaggtgagaaaggagagtagaagcttatttcaagttccagtccct
cctgatctacattctcctgtaattattagcctatgttaccatgtctgaac
agaaaatattggtgatgcccttgatcatgaatagctatcatgtctctgtt
ctggcttgctcctggatttttttttttccagtttcatgatgctgcatcac
tctgtcaccagtcctcactgttcgtttctatggaacatgaaatggtgaat
ggtccatcctttactgctgatactagtcattgctgaaacagccaccctaa
agatctcacagtctctctgtttataaacatttaagagtcaagtcatgaag
gctttctcccttaactacatgggatgatcagttgttttatctgttatttt
ttctgttattacttttgtccttaattgttagagaaaactttcatataaca
cagttactacataaatgccccctcccttcacatcttagaatgcctctggc
catctctgtagttgtacttgagaggtttaataaagtaccataagtttggg
aaaattagctttattgatataagcacttcctggagaatatttcttcacta
tacaaaaagctcatatttgatcattgttttctctatatctgtctatctct
ctcacatacattctgagtgcctgtgtgtgtgtgtgtgtccccagtgactt
ctgcatctgtagtagctggaataacatgtgtgtcacatgtacccccacca
cacacacacacacacacacacctctgccacgggcttcgttcaattctatt
ctcaatttccaaccttgtcagccatcttgatttcctcttctccaaacctc
aaagaaactgttaatgagtacccgagaatgaaaatgttgggcatatggaa
aaactgaaagacaatccatattgccataaaatggcctgcttttatctgga
aaagccttattatcactcacactcattccttctacatcctttacctcctt
ctctttgtctttctgtctgtctctcattctgtctctatcacacacacaca
cacaacacacacagagagagagagagggagagagagagagagagagagag
aaagagagactggctataaagaaagcagcatctgagggcatcaatggtaa
ttcgaacattttgtttgcttgggatcaagattcctttccatcaccattgt
cctggcaaatataagctgcaagtggaagtgttttagccagatactgctcc
accactctatggctaactaagcaggaatatcagcttcaactatgccatgg
aattcaagagaatattcaacctggaaaaattctaaccccaaacagcacct
cccaaaagatgactacagctcctgtaggaaatcatttaaccacaaattcc
aactccccttcactcctacagcctcagtcacacatctcaaagggctgatc
cttgaattgtgacaacctgacccacatcagcagcccagaggccagatggc
aacacaatgcctttccagcctactgggtaggaaaagggagggacaagcaa
ttgaatgattataattgaaatcctgtaatttattatttgtcaataactcc
tgccttgggggagttcccttcactccttagcaaatgctggcagccgcaat
atgtgaccacagacacctaaaacaccactgaaagcatttcattatgtggc
aacaatgatatggagaataagatctctctggagataagaagacatgccac
atctcagagttcactattcaacagcaaagaatttgaatagggggaaaaat
tccttagacttagggggaaaaattcctgagattctgagggtaaaaagcta
gcatgcaagtgggatcagccagactggcaggaagtggggcatgaaaaccc
aagaactatcctcctgtttgcagtataatgttacccctgcagttattaaa
ctgcacagatcaaataactatttgaactgagctgagctgagtcaagcaaa
gaaatgtacttcaatcatagaatagtagctgtcaggttgaaaagggcatc
tatcagacatctacccaatgttcctctacctaactcccatcctaattatc
cctgtctcatgttcatctccctgtaattgatgcaacctgatgcaacataa
agaggatttggagtgaaaagccctgagttggaggcctgggccttgacttt
ttaatttactagccataccacattggtccttctagtcttcagctacaatg
tgagcagattaaactagttgatgccaagattccatccagttcaaaattct
ctaggcaataatagggaactcactatctccaaaaataattatttgcatgt
ttggacaattctattaaaaagtcgtcacttacataaagccaaaacatggt
ttcttatagtttctacatatttatctgagttctacctgccttgagattat
atagtccaagtataatccctcttctatataacagccactcatatagccaa
agacagatattgcatctcccatccatctatccatccatccatccatccat
ccatcccactgagttctgtttgccactcatctagacaatcattctatcta
ctttactcagaacacataccaatttgtccatatccttactgaaaaatggt
catcagaactgaacaaagagctacagaaatagtttgataaatgctgagcc
aagtacctcaccacctattttgttccagctaatgtgtttattttaatgca
gcctaatatcacattatctttttggctatggcatcactctgccactgcca
caatattgctttttttactcatgtcttctaccctatccttgtaaagtgta
atttggaactggagtcagaattttactttttcaataatattcatttatca
gatttgagccattggtgcaacatgataaaatcttcttgaatcctcagtct
atcctaaaaatatttgccatctatccaaattttcacctaagttattgata
aaatacaatgagcacaacatggtcaggaaaaaagtcctagagcagaccac
taaacaataccctccatattgacataattctgcaagatttggggtctttt
ggccccaaagaaggtcatatccaagattgcctcattcttgacactctctc
aatgctaccttcttcactcactcatagtccatggtctagtctttctgctg
atcctgcaatgatttccacagttaagttcacccagacataggtactttga
gagaaatcactgtttaaaacattaaaaatataaataaggttaacaaaaaa
aggataaaagttcacacccagaacaatatgccatggtggaaataatgtgg
gagttggagtctgaataacctcagattaaatcctagttctgtaacttact
tgcttggtgaccttgggtgccttatcagtatcagttccctcatctctaaa
atagaggaataataatgacctcaaagggttgtcctgatgattaaaattaa
tacacgcaaaatcttagcacattcctagcacataatgggtatacaatata
gattactatcattattacatcatggtagagacaaaaatgcaataactact
ggcataaaaaaattagacccaagaagaaaaaaggagaaaatgaatatctg
tgttctacagatgttaaccaatactccacaaaagaaagaatcaatggtct
aataaggttaagaacgaggacattaaacacagttttaaaaactcgggcat
atttttatctctttgactctgaacaagattggcacacccttgcatccctt
atttgatcatgtcccaggcattgctgtagggcccaaggacacaaaggatg
actcagacccagtcaatgctagggtgagaagaaacacaggattcaaggca
ccagggggcagggggcacacatataaataagtaattaatataaaatatca
ccagtgctattgagaggtaaatacagagtatgtgagggaacactgataaa
tctggagatgtcaggtaaggctttatgcaggaggtggtaattttgaagaa
tcttaaaagataaagagaagttaaggatgagggtctctcaagtcaaggta
gtggaggatagcacgcaaagattttgaacagtagtggaccaatatgggct
cgtccaagaaatattgctcattctagagtgacttttccatgagatacagg
taataagagggacaattgaaaagatacagattcatgcctattgtaaaggg
gcttgtacgtcaggtaaaaaagtctgaaattctgcaggcaagaagaaacc
agcacagcgaatgacataagatggtaagccagcttaatagtagagagaga
ggctagttacgacacttttttttctttaccaatacccaattgaaagatgg
tcaggtccagaactaaaattatagcactggaggtagagaataggcctaca
taacaaaatctaagcctgagtataattgatacgatgtgacatgagcatga
ggggttaaggaaaaggagaaatgaaaatgactttgtctagcttgagacac
ccagtgtgtgggggcatcattaccaatatatgggcttctggaggatagta
cattttggtaggaggcacaatgtaagttcagttttcaacactatggattg
agataaccatgggacatccatatggagatgcaatctgctcaacataacag
tgtgtatatcataagacagaccagggataggagtcccctgtatatttatg
gcaataaaagaaaattcatggtttaagaaggaagagtatgtggaacatgt
ctctgatgccacgatgtaataaccttgaaaacaaagtaaaattacactaa
tgagtcttgcctaattaaactcatgctcctagtgatcaccacttctagtt
caattgttcacattcttgctctgctttgaaaaattaaaattaaatttgcc
tatcctctactgaccataatttctagaagacggcattcatctcatggcaa
gttcttcagtacccaaagatggaatacatagattaaaaaagaacatatat
gtagatgcttgtgatgttttcctatcataaattgaatttcaagttcttat
aaacgtattaatatgtcctactcttctagagacaaggatcaggaagtgta
tttatcaatagatatttaccaagcacctgtcaagccaaagtggggttaca
gaaaagtaggtatgggccctgcacacaaacaacctgtattagccaaaggg
acccttccataaaatttccaatatgtaaacccaaatttggaacttgctga
aacaagtacagatgagtacgtgaggaagctgggaagtaaacacaggttgc
tggagaaatagaggtggagatatgggtggatctaggtttggttaggaatg
aatcagaccatcccacagagggtggctcctccctgcatggggcctgctat
aaaagggccattatctcagccttcagtacccagcaggctccttcaggcta
cattctatttgct

As such, the invention provides nucleic acid constructs comprising a sequence selected from the group consisting of:
(i) a sequence exhibiting a degree of identity or homology to SEQ ID NO: 1; and/or
(ii) a promoter fragment having a sequence exhibiting a degree of identity/homology to a fragment or fragments of SEQ ID NO: 1.

The nucleic acid constructs of this invention may comprise a sequence exhibiting a degree of identity or homology to a human filaggrin promoter sequence derived from a clone of a human genome library. For example, suitable human filaggrin promoter sequences may be obtained from Bacterial Artificial Chromosome clone (BAC) libraries. An exemplary BAC clone is RP1-14N1.2 which comprises a sequence encompassing the entire human filaggrin locus. Specifically, BAC clone RP1-14N1.2 comprises a sequence which exhibits a degree of homology identity to sequences of the human filaggrin locus. The sequence of BAC clone RP1-14N1.2 comprises the following filaggrin sequences: ˜10 kb upstream of the transcription start site, the 15 bp exon 1 (partial 5′UTR) sequence and the first 18 by of intron 1 (ending at the Mlu1 restriction site).

The nucleic acid constructs of this invention may further comprise sequences which exhibit a degree of identity or homology to the human filaggrin gene or to one or more fragment(s) or portion(s) thereof. This additional filaggrin sequence will be referred to hereinafter as the “filaggrin component”. The filaggrin component may not comprise a sequence which represents a filaggrin coding sequence. In other words, the filaggrin component may not comprise a sequence which encodes a functional filaggrin protein.

A fragment of the human filaggrin gene may comprise between about y and about n−1 nucleotide bases of the human filaggrin gene. The term “y” may encompass a fragment comprising about 10 to about 600 nucleotides of the human filaggrin gene. For example, suitable “y” sized fragments may comprise about 20, 30, 40, 50, 100, 150, 200, 250, 300, 350, 400, 450, 460, 470, 480, 481, 482, 483, 484, 485, 490, 500, 550 or about 600 nucleotides. The term “n” represents the total number of nucleotides of the human filaggrin gene. As stated, it should be understood that the nucleotides of any filaggrin component of the construct may be derived from a single region or domain of the human filaggrin gene or from multiple different regions or domains of the human filaggrin gene.

The filaggrin component of the constructs described herein may comprise a sequence exhibiting a degree of identity or homology to a nucleotide sequence of exon 1 of the human filaggrin gene or a sequence or sequences exhibiting a degree of identity or homology to a fragment or portion of exon 1 of the human filaggrin gene. The filaggrin component of the nucleic acid constructs of this invention may comprise a sequence which exhibits a degree of identity or homology to all, or substantially all, of the sequence of exon 1. The sequence of exon 1 is given below as SEQ ID NO: 2.

SEQ ID NO: 2
CTTTTGGTGAACAAG

As such, and in addition to the filaggrin promoter element, the nucleic acids of this invention may comprise a filaggrin component, the filaggrin component comprising a sequence exhibiting a degree of identity or homology to SEQ ID NO: 2 or a fragment (for example a fragment comprising 5, 6, 7, 8, 9, 10, 11, 12, 13 or 14 bases) thereof.

Additionally, or alternatively, the filaggrin component may comprise or further comprise a sequence which exhibits a degree of identity or homology to the sequence of intron 1 of the human filaggrin sequence or a sequence or sequences which exhibit a degree of identity or homology to one or more fragments or portions of intron 1 of the human filaggrin gene. By way of example, the filaggrin component of the construct may comprise (in addition to any sequence exhibiting a degree of identity or homology to exon 1 of the human filaggrin gene) a sequence which exhibits a degree of identity or homology to 5′ and/or 3′ regions or domains of intron 1.

A sequence exhibiting a degree of identity or homology to a sequence of inton 1 of the human filaggrin gene is given as SEQ ID NO: 3.

SEQ ID NO: 3:
gtaagaaggaatacgcgt

A further sequence exhibiting a degree of identity or homology to a sequence of intron 1 of the human filaggrin gene is given as SEQ ID NO: 4.

SEQ ID NO: 4
atcttgtcatatggctaactggctttcagagaattgtgtcttagcaaatt
aatcatttattaagttagcttttggggaagtaattttcagtaaatttgta
taagtaaattttactgcttaaaaccagtcagcctttctaaattcttgttg
ctagcaatggcaagatcagcaagagggatgatcttacctagagcacactg
aggtctgtgagactactaagtccagctgtaagtgggcacaaggccaaaaa
atgggcaattttatttaagtcaggaaaactctattcatttgttttatgta
gatggtagagaaaagtttctcatgtccatcttccattcttaaagatggat
tacttagaggaaaatgtaggtttaaaatgtggaaaatgattgtaataata
taacctagaaaaaaagatgtataaaaaagtgcattttcatttcttctttc
tag

The filaggrin component of the nucleic acids described herein may comprise SEQ ID NO: 3 and/or SEQ ID NO: 4.

The filaggrin component may, in addition to comprising a sequence of SEQ ID NO: 2, may further comprise a sequence of SEQ ID NOS: 3 and/or 4 (or fragments of either).

In addition to any sequence derived from SEQ ID NO: 2, the filaggrin component may comprise the sequence of SEQ ID NO: 5 of a sequence derived therefrom. SEQ ID NO: 5 represents a combination of SEQ ID NOS: 3 and 4 and is therefore a sequence comprising domains/regions which each exhibit a degree of identity or homology to a part of intron 1 of the human filaggrin gene.

SEQ ID NO: 5
gtaagaaggaatacgcgtatcttgtcatatggctaactggctttcagaga
attgtgtcttagcaaattaatcatttattaagttagcttttggggaagta
attttcagtaaatttgtataagtaaattttactgcttaaaaccagtcagc
ctttctaaattcttgttgctagcaatggcaagatcagcaagagggatgat
cttacctagagcacactgaggtctgtgagactactaagtccagctgtaag
tgggcacaaggccaaaaaatgggcaattttatttaagtcaggaaaactct
attcatttgttttatgtagatggtagagaaaagtttctcatgtccatctt
ccattcttaaagatggattacttagaggaaaatgtaggtttaaaatgtgg
aaaatgattgtaataatataacctagaaaaaaagatgtataaaaaagtgc
attttcatttcttctttctag

As such, and in addition to the filaggrin promoter element (for example the sequence of SEQ ID NO: 1 or a promoter fragment thereof), the nucleic acids of this invention may comprise a filaggrin component, the filaggrin component may comprising one or more sequences selected from the group consisting of:

a sequence exhibiting a degree of identity or homology to SEQ ID NO: 2 or a fragment thereof;

a sequence exhibiting a degree of identity and/or homology to SEQ ID NO: 3 or a fragment thereof;

a sequence exhibiting a degree of identity and/or homology to SEQ ID NO: 4 or a fragment thereof; and

a sequence exhibiting a degree of identity and/or homology to SEQ ID NO: 5 or a fragment thereof.

In addition to any sequences exhibiting a degree of identity or homology to sequences of exon 1 and/or intron 1, the filaggrin component may comprise, or further comprise a sequence which exhibits a degree of identity or homology to the sequence of exon 2 of the human filaggrin sequence. Alternatively, the filaggrin component may comprise, or further comprise a sequence or sequences which exhibit a degree of identity or homology to one or more fragments or portions of exon 2 of the human filaggrin gene. By way of example, the filaggrin component of the construct may comprise (in addition to any sequence exhibiting a degree of identity or homology to exon 1 and/or intron 1 of the human filaggrin gene) a sequence which exhibits a degree of identity or homology to a 5′ region or domain of exon 2. The filaggrin component may comprise or further comprise a sequence which exhibits a degree of identity or homology to the 5′UTR of exon 2 of the human filaggrin gene.

A sequence which exhibits a degree of identity or homology to the sequence of exon 2 of the human filaggrin gene (in particular a 5′ region or domain of exon 2) is given as SEQ ID NO: 6.

SEQ ID NO: 6
gttcacatttattgccaaaagctt

A nucleic acid construct of this invention may comprise a sequence exhibiting a degree of identity or homology to SEQ ID NO: 7:

SEQ ID NO: 7
ctcgagtcagtgtctggcagtgaatgagctacaaattgttttcatattgc
ttacctgaaggccagtgcttgtttagctgctgaagaaaaatagaaacctt
atggcatttagaacatagtttattctttaagtgcagaagtgtgtgactta
acccttgactggcatggtcttagctcctgtttacaatttggtatcttact
gccacaaagagtctgttctatcagtcttacattctctattttcacatcaa
tgctggccagttgtgtctaaacactgcaaaagggagggtatataacaaga
tatgtctgacttcctgagccatcatggctgggaactcagtttttaagatt
tatctagggtccatttggccaagattggggtggggagggtctgttcagtc
agttgagggctttaagatttatttttagtttacaggaggaaccatgttca
gactaccaaatagtttccaatgctgagcagggctagggccacagacacac
actcctgtttccatagaaattcctcaggcaggcctcatgtacttagccag
gaccctgctcttgcccccaaaggaatttatccttttaaaagtataatatt
cctcttatctgactgttctactgcacagtggggtaaccacagttaacaat
atcatatactatatttcaaaatagctattaatagaagagagaattttgaa
tattctcaccataaagaaatgacaaatgtttgaagtgattgatatgctaa
ttaccctgatttgatcattacataatgtaaacatgtatagaaacatcact
taaatataaacatgcacaattactagatgtcagttaaaaacaaaataaaa
cttagaaaagtataatgtgatgatactggttcagggatctgatctttaat
ccgaaggaaaagaggagacatgaaagaactgaggggagggcttgttattc
ctcttcctcccctttctctattaccacccttgcattaacatgcccctggt
tatcttgctatgttttttatatttattatgaatgcagaacacagcaaaaa
gtaaatagcaattatatcagttctaattctgcttcaagtaatagaacaac
tcaaagcttattaaattaaagggagaatgtattggttcacgtaattgaaa
agtcttgaaatggggctagcaggctgtactcactccaaggctcaaagtat
atcatcaagattgggtgagtctctcaatctctcagctctgcttttccctc
tgttgactacagttttgacatgtagtcatgagatggctgcagcagcttgg
cctacattcttccctgttcaaatctcaaggtgcaggggccaaggggttgg
ggggagaggtggaggaggaatgctatgtctttattggtagttcacacaat
agtcctgagattcactctgccttagacatcccaggtcacatgcttaccca
tccctgtccactaacatccaagcaacatcttcaccctcataagtcaaaga
taaactcaccccctcaccctgaccacaaggagtaagaatgggcaatggtt
ggattcttaaatgaaaatcagggactgttgccagaaaaaaaaaaaagtgg
aatgtatacaatggaggaaaacaataaatgtactagagcaatttttgcta
ctttttttgtagtatttgtattcaacattcaatattctcctgaagctact
cttttcctaaaacagatgaaaaccttctccccgatcttctagagatgatc
acactgaaccctaaaggacatttaaaaacctccataatcacacagagagg
gactcaattaaaaaaaattcttggaaaagaaaaaggaaaagatgtatgtt
tcttgctcttctctcttgatggaaacaaacacacatgcaaaaattcaatt
gcaataagtgtctcatattgagtttaccaattctgtaagacttcatagtt
atataaaactaataattaccatttactgaataccttgctatcatgctagg
caaagtgtcagaccattgacatataattcatttaatatactgaagaacta
tcaaaatgaagtattattatctttactctatagatgaggaaacagaccca
gaaaggttaagaaatttgctcaaagtcatagagctggcaactggaggagc
cagtagtcaaaggcaagtctgtgaggtgtgtgagattgtgctctcaacac
tacaccatggattttgtgatcttggctctaattagggggccaatatggtg
gacatggtgttatttgaatttaaccatttttcatatctcatatgaaaagt
tcctagaattaaaaatttcatgaagaaaacaaaaaatagctgaggatttc
tagatgcatactgtgaaaagaaatgcaaatattaatagtaaccatgttct
atgaattagtttaagcatatacatgaaatgctgttagagtacacgttgca
agagtaactaactgatggcctgattagaaaaatgatagaatgaggtaaag
aaagaggtagattctgggaaatatggattaaaatgatgaaacaaaaaatg
agaaaaaaactaccttaccttctcaagtattgccatagaagagcagaagt
catcctgagcaaagctaaaggcaaaaaacctaggctccagactgatacac
tggcttgataatccacactgtgagatgtcaccaaaagttgtctgatccag
ctgtggggacttggctccaagttgtctggatatcctaaaataatataaat
cccttgaaacaagtgctctaagaaatggaggaagccatactttagggaat
gctgataaaatgagtggaaatgttggtggtgtgagtccacctctacctca
acaaggtcaccactgatgtgagaatccatgaggttaacaaaaacgagcat
cattagtctcataaattaacacttgaaggactttggagccttgggtctca
gtgattcttctggaaatatggtagtttagggtctttaataatttttttaa
cttttgttatgagcagaaattgtaaataacatgtaaatacaactccagag
tggttctgatgttcaccttctgaactcatctagttctcaaactactagat
tatgcaacccttgtgctcctacagctaaaccacatatactattctgagcc
ctcccctaactacacctgggtaagaggagtgtggtgtagtctggtgttgt
gctataccaatagtcatggagatatcaatcaaacatctgcctaatctaca
aggtgtaggtggtgaattatgaagaatgagtaaccaggagacttacgatg
agagaataaagtctaagttgataaaaagttagtgctgagtagatagaggg
gtcttcaggaattggaagcttggtgagggtccccatcatccagaataagt
actggtcttcagcaggaagaatttaagaatagaagttacctaaagctcat
gaattagtatcagttcaattatgctggtaatctgattttgatctttctca
gttatgataaaagtctctgcctggtcatgatataaaccttctttgttctt
tagtcttccctacttacaagattaaagtcctggcctaaaatatgagccct
aaggtcctgctacctagtgacatgatattgttgataccaactctccatta
cccactgcctactgaaatctcctattcctgccttctccatcattgagttc
tgtccacctgtcagagtctacttcattgggttgccctccctaacctgccc
aacaccaactgcttgccaggccgctaacctattccctcttgttctgcacc
ttctctcatagtccacttccaatgcatcaccaagatctgcctctaaaatg
tatcttctcttcaatcccactgccaatgccttagtttgagattttactat
ctcttccttcttttatataaattgcctgtatccagctcaatccgccatcc
atacttccagtagttacatttactgaatgaaaatctggtcctgtcacttt
tccacttaaaaacccttcagttgccacccaatataaataatatatacatc
acttagaggcttccaaggcttccaagccctttccatatgacctttcaggc
ctgcctctccctgatctttcccatacaatccaataagtgcttttattgtt
ctttaatgttctttaatgtcctttaatgttcagctcaaatattacctccc
cttgacctctctaaaagaaaacagcttgcttcctcaagtaaagctattta
ctaatacctaaggtattcctaaaggtatttacaaatacctttattataac
acatatcatactgtttatatgtatgctttcttcattagattataaactct
tggaggacaaggagcatgccttcttcatttacagttctgtgacaagcatg
gtgactggcaagtagtcactggcaagaaatgtttcttgaatgaataaatg
atccctaaatactgtgacctatctcttagtctgaatttccctcagttacc
tgcagaaattttccctctggaatatattcttgtttatctatctgtctacc
tgtctgtctgtctgtctattctatctatctataatctccctatatacaaa
ggaaacaggtgagaaaggagagtagaagcttatttcaagttccagtccct
cctgatctacattctcctgtaattattagcctatgttaccatgtctgaac
agaaaatattggtgatgcccttgatcatgaatagctatcatgtctctgtt
ctggcttgctcctggatttttttttttccagtttcatgatgctgcatcac
tctgtcaccagtcctcactgttcgtttctatggaacatgaaatggtgaat
ggtccatcctttactgctgatactagtcattgctgaaacagccaccctaa
agatctcacagtctctctgtttataaacatttaagagtcaagtcatgaag
gctttctcccttaactacatgggatgatcagttgttttatctgttatttt
ttctgttattacttttgtccttaattgttagagaaaactttcatataaca
cagttactacataaatgccccctcccttcacatcttagaatgcctctggc
catctctgtagttgtacttgagaggtttaataaagtaccataagtttggg
aaaattagctttattgatataagcacttcctggagaatatttcttcacta
tacaaaaagctcatatttgatcattgttttctctatatctgtctatctct
ctcacatacattctgagtgcctgtgtgtgtgtgtgtgtccccagtgactt
ctgcatctgtagtagctggaataacatgtgtgtcacatgtacccccacca
cacacacacacacacacacacctctgccacgggcttcgttcaattctatt
ctcaatttccaaccttgtcagccatcttgatttcctcttctccaaacctc
aaagaaactgttaatgagtacccgagaatgaaaatgttgggcatatggaa
aaactgaaagacaatccatattgccataaaatggcctgcttttatctgga
aaagccttattatcactcacactcattccttctacatcctttacctcctt
ctctttgtctttctgtctgtctctcattctgtctctatcacacacacaca
cacaacacacacagagagagagagagggagagagagagagagagagagag
aaagagagactggctataaagaaagcagcatctgagggcatcaatggtaa
ttcgaacattttgtttgcttgggatcaagattcctttccatcaccattgt
cctggcaaatataagctgcaagtggaagtgttttagccagatactgctcc
accactctatggctaactaagcaggaatatcagcttcaactatgccatgg
aattcaagagaatattcaacctggaaaaattctaaccccaaacagcacct
cccaaaagatgactacagctcctgtaggaaatcatttaaccacaaattcc
aactccccttcactcctacagcctcagtcacacatctcaaagggctgatc
cttgaattgtgacaacctgacccacatcagcagcccagaggccagatggc
aacacaatgcctttccagcctactgggtaggaaaagggagggacaagcaa
ttgaatgattataattgaaatcctgtaatttattatttgtcaataactcc
tgccttgggggagttcccttcactccttagcaaatgctggcagccgcaat
atgtgaccacagacacctaaaacaccactgaaagcatttcattatgtggc
aacaatgatatggagaataagatctctctggagataagaagacatgccac
atctcagagttcactattcaacagcaaagaatttgaatagggggaaaaat
tccttagacttagggggaaaaattcctgagattctgagggtaaaaagcta
gcatgcaagtgggatcagccagactggcaggaagtggggcatgaaaaccc
aagaactatcctcctgtttgcagtataatgttacccctgcagttattaaa
ctgcacagatcaaataactatttgaactgagctgagctgagtcaagcaaa
gaaatgtacttcaatcatagaatagtagctgtcaggttgaaaagggcatc
tatcagacatctacccaatgttcctctacctaactcccatcctaattatc
cctgtctcatgttcatctccctgtaattgatgcaacctgatgcaacataa
agaggatttggagtgaaaagccctgagttggaggcctgggccttgacttt
ttaatttactagccataccacattggtccttctagtcttcagctacaatg
tgagcagattaaactagttgatgccaagattccatccagttcaaaattct
ctaggcaataatagggaactcactatctccaaaaataattatttgcatgt
ttggacaattctattaaaaagtcgtcacttacataaagccaaaacatggt
ttcttatagtttctacatatttatctgagttctacctgccttgagattat
atagtccaagtataatccctcttctatataacagccactcatatagccaa
agacagatattgcatctcccatccatctatccatccatccatccatccat
ccatcccactgagttctgtttgccactcatctagacaatcattctatcta
ctttactcagaacacataccaatttgtccatatccttactgaaaaatggt
catcagaactgaacaaagagctacagaaatagtttgataaatgctgagcc
aagtacctcaccacctattttgttccagctaatgtgtttattttaatgca
gcctaatatcacattatctttttggctatggcatcactctgccactgcca
caatattgctttttttactcatgtcttctaccctatccttgtaaagtgta
atttggaactggagtcagaattttactttttcaataatattcatttatca
gatttgagccattggtgcaacatgataaaatcttcttgaatcctcagtct
atcctaaaaatatttgccatctatccaaattttcacctaagttattgata
aaatacaatgagcacaacatggtcaggaaaaaagtcctagagcagaccac
taaacaataccctccatattgacataattctgcaagatttggggtctttt
ggccccaaagaaggtcatatccaagattgcctcattcttgacactctctc
aatgctaccttcttcactcactcatagtccatggtctagtctttctgctg
atcctgcaatgatttccacagttaagttcacccagacataggtactttga
gagaaatcactgtttaaaacattaaaaatataaataaggttaacaaaaaa
aggataaaagttcacacccagaacaatatgccatggtggaaataatgtgg
gagttggagtctgaataacctcagattaaatcctagttctgtaacttact
tgcttggtgaccttgggtgccttatcagtatcagttccctcatctctaaa
atagaggaataataatgacctcaaagggttgtcctgatgattaaaattaa
tacacgcaaaatcttagcacattcctagcacataatgggtatacaatata
gattactatcattattacatcatggtagagacaaaaatgcaataactact
ggcataaaaaaattagacccaagaagaaaaaaggagaaaatgaatatctg
tgttctacagatgttaaccaatactccacaaaagaaagaatcaatggtct
aataaggttaagaacgaggacattaaacacagttttaaaaactcgggcat
atttttatctctttgactctgaacaagattggcacacccttgcatccctt
atttgatcatgtcccaggcattgctgtagggcccaaggacacaaaggatg
actcagacccagtcaatgctagggtgagaagaaacacaggattcaaggca
ccagggggcagggggcacacatataaataagtaattaatataaaatatca
ccagtgctattgagaggtaaatacagagtatgtgagggaacactgataaa
tctggagatgtcaggtaaggctttatgcaggaggtggtaattttgaagaa
tcttaaaagataaagagaagttaaggatgagggtctctcaagtcaaggta
gtggaggatagcacgcaaagattttgaacagtagtggaccaatatgggct
cgtccaagaaatattgctcattctagagtgacttttccatgagatacagg
taataagagggacaattgaaaagatacagattcatgcctattgtaaaggg
gcttgtacgtcaggtaaaaaagtctgaaattctgcaggcaagaagaaacc
agcacagcgaatgacataagatggtaagccagcttaatagtagagagaga
ggctagttacgacacttttttttctttaccaatacccaattgaaagatgg
tcaggtccagaactaaaattatagcactggaggtagagaataggcctaca
taacaaaatctaagcctgagtataattgatacgatgtgacatgagcatga
ggggttaaggaaaaggagaaatgaaaatgactttgtctagcttgagacac
ccagtgtgtgggggcatcattaccaatatatgggcttctggaggatagta
cattttggtaggaggcacaatgtaagttcagttttcaacactatggattg
agataaccatgggacatccatatggagatgcaatctgctcaacataacag
tgtgtatatcataagacagaccagggataggagtcccctgtatatttatg
gcaataaaagaaaattcatggtttaagaaggaagagtatgtggaacatgt
ctctgatgccacgatgtaataaccttgaaaacaaagtaaaattacactaa
tgagtcttgcctaattaaactcatgctcctagtgatcaccacttctagtt
caattgttcacattcttgctctgctttgaaaaattaaaattaaatttgcc
tatcctctactgaccataatttctagaagacggcattcatctcatggcaa
gttcttcagtacccaaagatggaatacatagattaaaaaagaacatatat
gtagatgcttgtgatgttttcctatcataaattgaatttcaagttcttat
aaacgtattaatatgtcctactcttctagagacaaggatcaggaagtgta
tttatcaatagatatttaccaagcacctgtcaagccaaagtggggttaca
gaaaagtaggtatgggccctgcacacaaacaacctgtattagccaaaggg
acccttccataaaatttccaatatgtaaacccaaatttggaacttgctga
aacaagtacagatgagtacgtgaggaagctgggaagtaaacacaggttgc
tggagaaatagaggtggagatatgggtggatctaggtttggttaggaatg
aatcagaccatcccacagagggtggctcctccctgcatggggcctgctat
aaaagggccattatctcagccttcagtacccagcaggctccttcaggcta
cattctatttgctcttttggtgaacaaggtaagaaggaatacgcgtatct
tgtcatatggctaactggctttcagagaattgtgtcttagcaaattaatc
atttattaagttagcttttggggaagtaattttcagtaaatttgtataag
taaattttactgcttaaaaccagtcagcctttctaaattcttgttgctag
caatggcaagatcagcaagagggatgatcttacctagagcacactgaggt
ctgtgagactactaagtccagctgtaagtgggcacaaggccaaaaaatgg
gcaattttatttaagtcaggaaaactctattcatttgttttatgtagatg
gtagagaaaagtttctcatgtccatcttccattcttaaagatggattact
tagaggaaaatgtaggtttaaaatgtggaaaatgattgtaataatataac
ctagaaaaaaagatgtataaaaaagtgcattttcatttcttctttctagg
ttcacatttattgccaaaagctt

The constructs provided by this invention may comprise or further comprise a nucleic acid sequence which is operatively linked to the filaggrin promoter element of the nucleic acid construct. The sequence operatively linked to the filaggrin promoter element may encode a reporter gene.

The reporter sequence may encode a gene or peptide/protein, the expression of which can be detected by some means. Suitable reporter sequences may encode genes and/or proteins, the expression of which can be detected by, for example, optical, immunological or molecular means. Exemplary reporter sequences may encode, for example, fluorescent and/or luminescent proteins. Examples may include sequences encoding firefly luciferase (Luc: including codon-optimised forms), green fluorescent protein (GFP), red fluorescent protein (dsRed). One of skill will be familiar with the range of commercially available vectors which encode reporter gene sequences. Vectors of this type may further comprise one or more sites designed to accept heterologous sequences which may comprise genes capable of directing the expression of the reporter gene sequence. An exemplary vector system may be the luc2P/Puro vectors produced by Promega. For example, the pG4.21[luc2P/Puro] vector is a basic vector with no promoter and multiple cloning regions into which a promoter of choice may be cloned. Using such a vector, the luc2P gene can be placed under the transcriptional control of a human filaggrin promoter sequence.

The nucleic acid constructs of this invention may comprise a nucleic acid sequence encoding:
(i) a human filaggrin promoter sequence;
(ii) a filaggrin component (for example as defined above); and
(iii) a nucleic acid sequence operatively linked to the human filaggrin promoter sequence.

As stated, the nucleic acid sequence operatively linked to the human filaggrin promoter sequence may not be a sequence encoding a functional, complete or wild-type/native human filaggrin gene. Moreover, the filaggrin component may not comprise a sequence which encodes a functional filaggrin protein.

Reading 5′ to 3′, the nucleic acid construct of this invention may comprise (i), (ii) and (iii) [as defined above] in that order—i.e., filaggrin component (ii) is between promoter sequence (i) and the nucleic acid sequence (iii) operatively linked to (i).

The human filaggrin promoter sequence may comprise a sequence exhibiting a degree of identity or homology to SEQ ID NO: 1.

The filaggrin component may comprise (1) a sequence exhibiting a degree of identity to exon 1 (SEQ ID NO: 2); (2) (i) a 5′ region (SEQ ID NO: 3) and a 3′ region (SEQ ID NO: 4) of intron 1 of the human filaggrin gene; or (ii) a sequence comprising regions or domains which each exhibit a degree of identity or homology to 5′ and 3′ regions of intron 1 of the human filaggrin gene (SEQ ID NO: 5); and (3) exon 2 (SEQ ID NO: 6) of the human filaggrin gene.

SEQ ID NO: 7 is a sequence which comprises (5′ to 3′) the sequence of SEQ ID NO: 1, the sequence of SEQ ID NO: 2, the sequence of SEQ ID NO: 3, the sequence of SEQ ID NO: 4 (SEQ ID NOS 3 and 4 together being the sequence of SEQ ID NO: 5) and SEQ ID NO: 6.

The nucleic acid sequence operatively linked to the filaggrin promoter element may encode a reporter gene sequence.

The nucleic acid construct of this invention may be as shown in the Figures of this specification and as substantially described herein.

In a second aspect, the present invention provides a non-human transgenic animal comprising a nucleic acid sequence according to the first aspect of this invention.

The transgenic non-human animal may be a rodent, for example a mouse, rat, rabbit, guinea pig, hamster or the like. The invention may provide a transgenic mouse.

In a third aspect, there is provided a method of making the non-human transgenic animal of the second aspect of this invention, said method comprising introducing a nucleic acid according to the first aspect of this invention into the germline of a non-human animal.

One of skill will appreciate that there are many ways in which nucleic acid sequences may be introduced into the germline of non-human animals and all such methods may be applied here. For example, a nucleic acid sequence (for example a construct according to the first aspect of this invention) may be introduced into the genome of suitable non-human embryonic stem cells. Using embryonic stem cell gene targeting techniques which may exploit, for example, homologous recombination events, it is possible to introduce a nucleic acid sequence into the genome of an embryonic stem cell. In this way, the genome of murine embryonic stem cells may be modified so as to include a nucleic acid sequence according to the first aspect of this invention.

Where the non-human transgenic animal is a mouse, a nucleic acid sequence according to the first aspect of this invention may be introduced into a genomic locus which permits generalised expression. For example, a nucleic acid of this invention may be targeted to the ROSA26 locus of the murine genome. As such, the ROSA26 locus of a transgenic mice according to this invention may comprise a nucleic acid sequence according to claim 1 (or as described elsewhere in this specification).

The present invention may provide a transgenic mouse comprising cells, the genetic material (genome) of the cells being modified such that the ROSA26 locus comprises a nucleic acid sequence encoding a nucleic acid construct of this invention.

Transgenic rodents (for example mice) produced in accordance with the methods described herein, exhibit localised expression of the reporter gene under the control of the filaggrin promoter element. This is surprising as nucleic acids introduced into the murine ROSA26 are generally widely expressed. The inventors have observed that reporter gene expression from the constructs described herein, is predominantly localised to the footpad epidermis. Additionally, the inventors have noted that reporter gene expression is detected in the granular layer of the epidermis—mirroring the expression profile of filaggrin in humans.

These features are particularly advantageous as they permit live, in vivo imaging of filaggrin promoter activity. Additionally, the murine footpad epidermis is a tissue which most closely resembles human skin and as such, the non-human (murine) transgenic animals of this invention provide a valuable model which may be used to test compounds for potential use in the treatment of human diseases associated with filaggrin expression. The localised footpad expression avoids the requirement for removing fur or other obstructive tissues, structures prior to imaging. The localised (footpad) expression of the nucleic acid constructs of this invention, ensures that the non-human transgenic animals may be used in left/right comparison studies in which control agents are applied to certain regions expressing the nucleic acid construct (for example the left or right footpad(s) or the fore or hind footpads) and test agents applied to the other regions.

In view of the above, the present invention provides methods of identifying agents potentially useful in the treatment or prevention of various diseases—in particular diseases which affect the skin and/or cells/tissues thereof. Such methods may generally be referred to as “methods of identifying therapeutic agents” and agents identified by such methods are potentially useful in the treatment and/or prevention of a disease.

A method of identifying a therapeutic agent may comprise:

contacting a non-human transgenic animal of this invention with an agent to be tested;

identifying modulation of reporter gene expression;

wherein modulation of reporter gene expression indicates that the test agent might be useful in the treatment of a disease.

The method of identifying a therapeutic agent may be exploited in order to identify agents potentially useful in the treatment of disorders or diseases of the skin (including disorders or diseases which affect the cells and/or tissues of the skin). The method of identifying a therapeutic agent may be used to identify agents potentially useful in the treatment of filaggrin based diseases. A “filaggrin based disease” may be any disease and/or condition caused or contributed to by filaggrin gene expression and/or the expression of mutated (variant) filaggrin genes and/or proteins. For example, filaggrin based diseases may include ichthyosis vulgaris (IV), atopic dermatitis and eczema. One of skill will appreciate that diseases of this type may stem from aberrant filaggrin expression and/or the expression of mutant/variant filaggrin genes (for example, the expression of truncated filaggrin proteins).

Agents which modulate reporter gene expression in the non-human transgenic animals described herein may find utility in the treatment of filaggrin based diseases as described above. Modulation of reporter gene expression may be detected as any increase or decrease in reporter gene expression in comparison to the level of reporter gene expression occurring in a transgenic animal not contacted with or exposed to, the test agent. Accordingly, test agents which are found to increase or decrease reporter gene expression may be useful in the treatment of filaggrin based diseases.

Suitable test agents may comprise, for example small organic molecules, amino acids, peptides, proteins, antibodies (or antibody fragments), carbohydrates, nucleic acids and the like. Additionally, the (non-human) transgenic animals provided by this invention may be used to test the potential utility of cutaneous delivery of gene silencing technologies, in the treatment of filaggrin based diseases. For example small interfering RNA (siRNA) or antisense molecules may be used as test agents.

It should be understood that any of the test agents described herein may used in isolation or together with one or more other test agents.

The test agents may be applied by any suitable means. The test agents may be applied topically and/or parenterally by injection. Test agents administered parenterally, may be administered by sub-cutaneous, intra-peritoneal, intra-muscular or intra-venous injection. Test agents may also be administered orally. Additionally, or alternatively, a test agent may be delivered using implanted osmotic mini-pumps. Pumps of this type are known in the art and a miniature infusion pumps which permit for the continuous dosing in, for example, mice and rats. Test agents may also be administered by periorbital delivery.

The step of contacting a test agent with a non-human transgenic animal of this invention may comprise the step of applying a test agent to one or more of the footpads of the transgenic animal.

The method of identifying a therapeutic agent, may further comprise the step of comparing the results with the results of a control experiment in which a test agent is not used. As explained above, the present invention represents a distinct advantage over the prior art as both test and control experiments can be conducted simultaneously (or together) in a single non-human transgenic animal. For example, the step of contacting a test agent with a non-human transgenic animal may comprise applying a test agent to one or more of the footpad(s) of the non-human transgenic animal. A control experiment may be conducted on one or more of the other footpad(s) of the same animal. Once the experiment is complete, the expression of reporter gene in the footpads contacted with test agent may be compared to the expression of reporter gene in the footpads used for the control experiments.

In a fourth aspect, the present invention provides a vector encoding the nucleic acid construct if this invention. The vector may take the form of a plasmid encoding one or more cloning sites.

In a fifth aspect, the present invention provides a host cell transformed with the nucleic acid of the first aspect of this invention or with a vector according to the second aspect of this invention.

The term “host cell” may encompass any embryonic or adult (somatic) cell. The cell may be a mammalian cell, for example a rodent cell. The cell may be a stem cell, for example an embryonic stem cell. The cell may not be a human embryonic stem cell. The cell may be a rodent, for example, murine embryonic stem cell.

### DETAILED DESCRIPTION

The present invention will now be described in detail with reference to the following Figures which show:

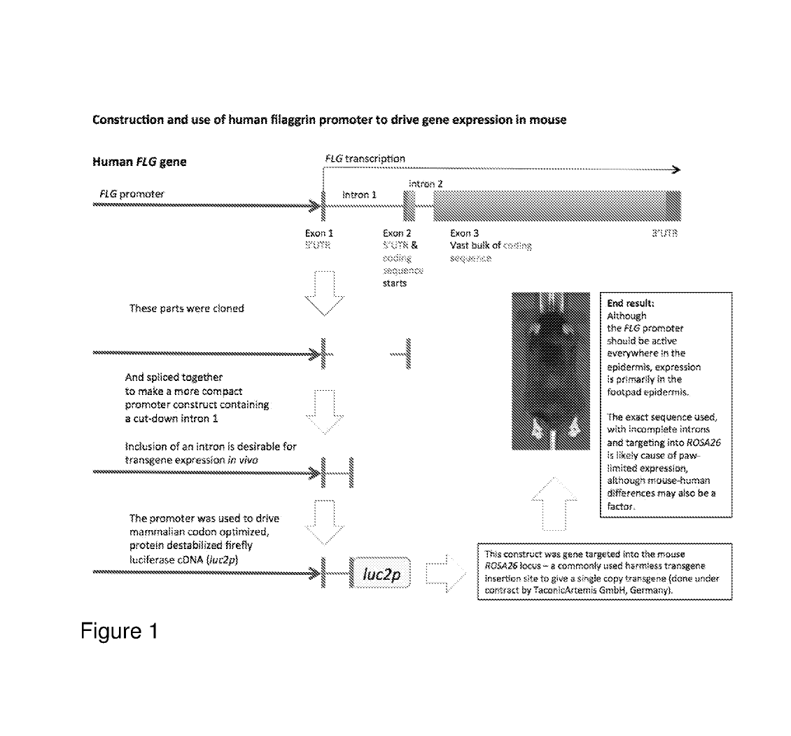

FIG. 1: Schematic of how the FLG-10K promoter construct was made and used to generate transgenic mice (not to scale).

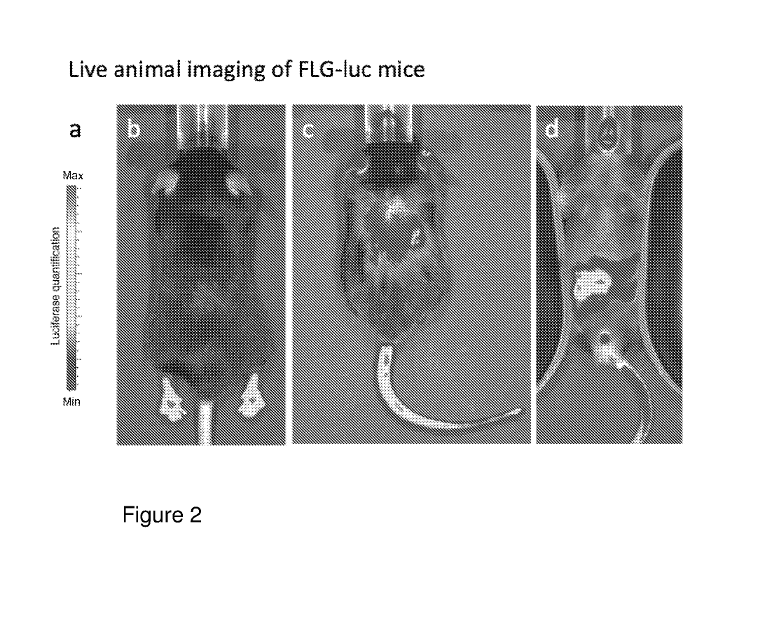

FIG. 2: Live animal imaging (under anaesthetic) shows very strong luciferase expression in footpad and much lower levels elsewhere in the epidermis.

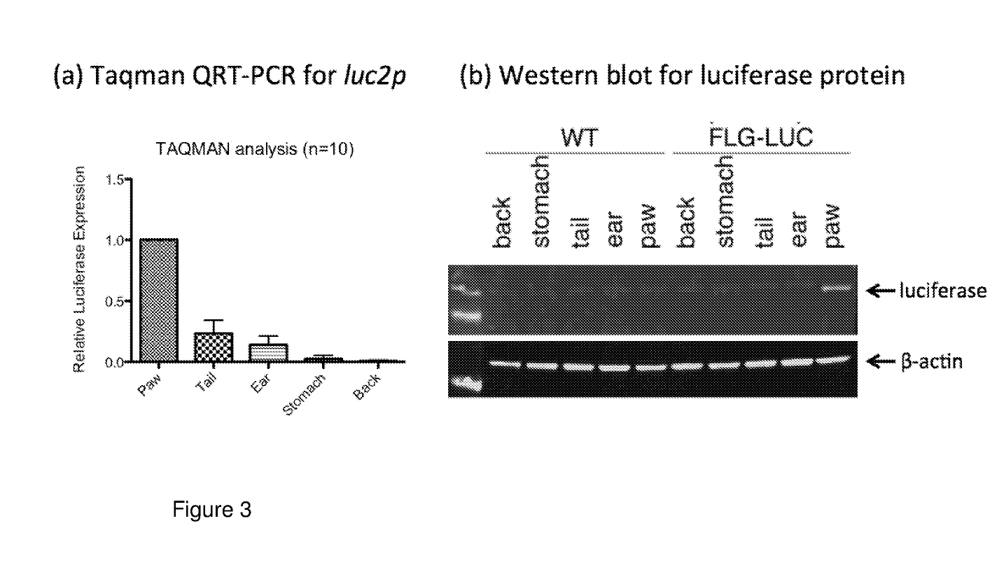

FIG. 3: Regional expression of luciferase in the FLG-luc2p mouse.

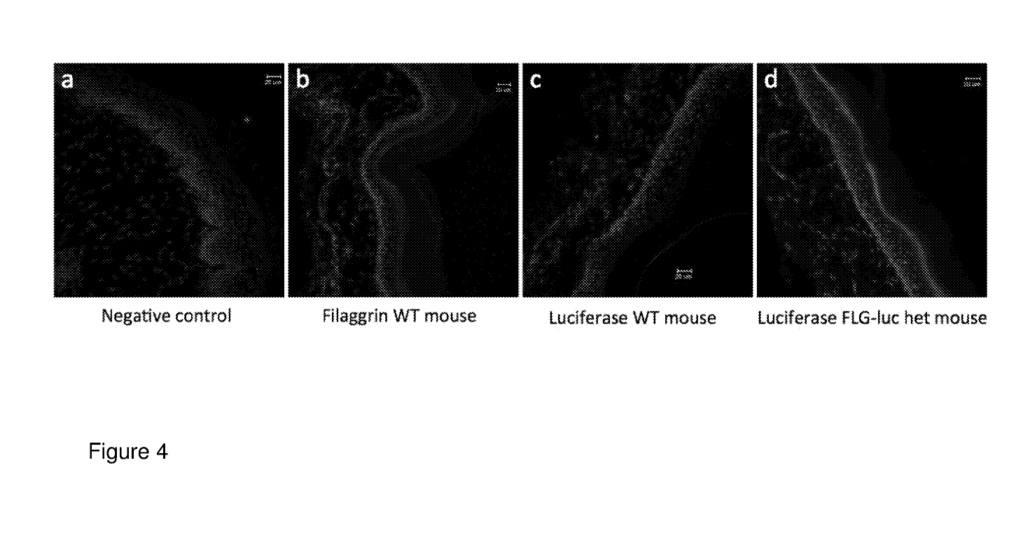

FIG. 4: Immunofluorescence localisation of luciferase within mouse footpad epidermis. (a) Negative control stained only for DNA to reveal nuclei (blue; DAPI stain). (b) Endogenous filaggrin staining (red). (c) Wild-type mouse epidermis has no luciferase expression. (d) In the FLG-luc mouse, luciferase is localised to the granular layers.

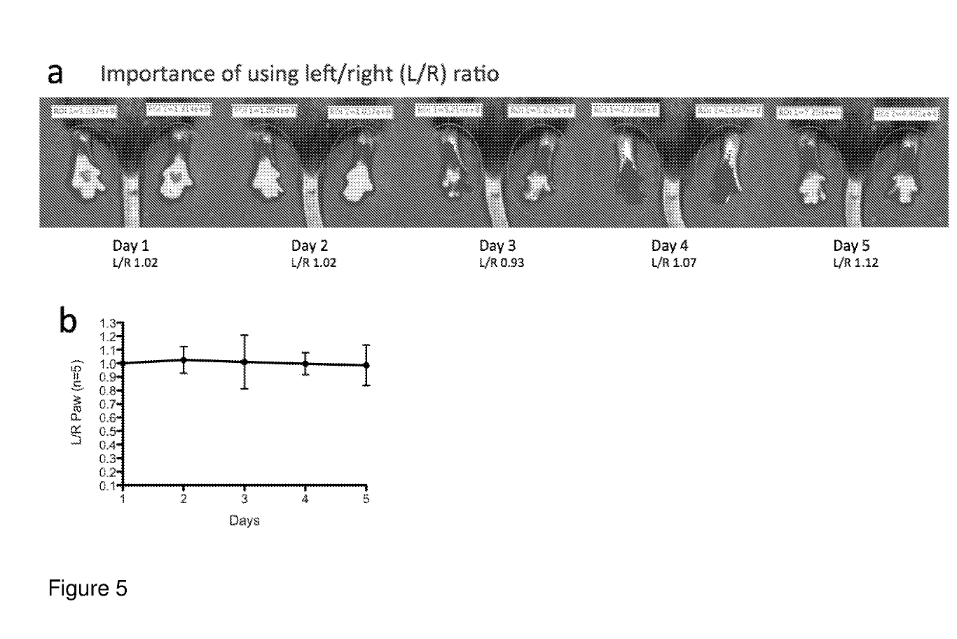

FIG. 5: The importance of using left-to-right ratio analysis in using FLG-luc2p mice to assess delivery of drugs or gene silencing agents to the footpad epidermis. (a) IVIS200 imaging of the same mouse on 5 consecutive days shows that while total gene expression varies from day-to-day expression remains consistent between the left and right paws. (b) Graphical analysis of left/right ratio over 5 days (n=5 mice).

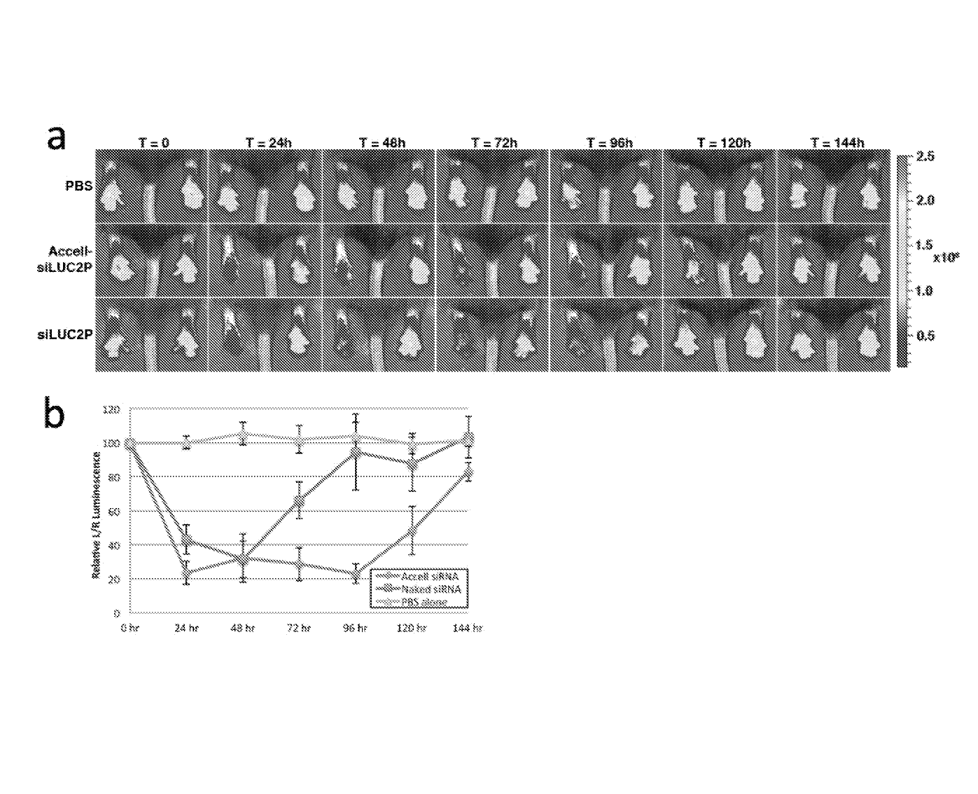

FIG. 6: Testing the usefulness of FLG-luc2p mice to assess epidermal delivery of drugs or gene silencing agents. (a) The left and right footpads of control mice were treated with vehicle only (phosphate buffered saline, PBS). (b) left paw was injected with either Accell siRNA or native siRNA targeting the luc2p mRNA and the right paw was treated with the corresponding native siRNA or Accell siRNA non-targeting control siRNA (NSC4, an inverted bacterial lacZsequence).

### MATERIALS & METHODS

### Nucleic Acid Construct (see FIG. 1)

A 10,146 by human filaggrin promoter fragment was cloned from a genomic bacterial artificial chromosome (BAC) clone encompassing the entire human locus using recombineering. This clone covers the region from ˜10 kb upstream of the transcription start site, all of the 15 by exon 1 (partial 5′UTR), and ends at an Mlu I restriction site just 18 by inside intron 1.

A 483 by fragment containing the last 459 by of intron 1 and the start of exon 2 (covering the remainder of the 5′UTR was generated by PCR and sequence-verified. This fragment had artificial restriction sites added to allow cloning and also the ATG of exon 2 was mutated out so that translation will start from the first Kozak sequence and ATG placed downstream.

The two fragments were ligated together via their Mlu I sites (within intron 1) to make a construct that consists of >10 kb upstream promoter sequence and a cut-down intron 1. The whole fragment is flanked by an Xho I site upstream and a HinD III site downstream and consists of 10,623 by of DNA. This construct was designated “FLG-10K” for convenience.

The Xho I-HinD III fragment was cloned into pGL4.21 so that the FLG-10K promoter drives luc2p expression. Luc2p encodes the mammalian codon-optimized, protein destabilized firefly luciferase gene.

### FLG 10K Promoter—Primer/Sequence Details

### Recombination Primers

Right arm:
Human Genome Build 18 (hg18), chromosome 1:
150,574,362-150,574,413
ggt aag caa tat gaa aac aat ttg tag ctc att cac
tgc cag aca ctg act cga gaC aac tta tat cgt atg
ggg c
ggtaagcaatatgaaaacaatttgtagctcattcactgccagacactgac
tcgagaCaacttatatcgtatggggc
Left arm:
Human Genome Build 18 (hg18), chromosome 1:
150,564,275-150,564,328
tcc ttc agg cta cat tct att tgc tct ttt ggt gaa
caa ggt aag Aag gaa tac gcg tta cgc ccc gcc ctg
cca c
GTGGCAGGGCGGGGCGTAACGCGTATTCCTTCTTACCTTGTTCACCAAAA
GAGCAAATAGAATGTAGCCTGAAGGA
10 K fragment is Human Genome Build 18 (hg18),
chromosome 1: 150,564,275-150,574,413
>hg18_dna range=chr1:150564275-150574413
5′pad=0 3′pad=0 strand=-repeatMasking=none

Cloned by recombineering, with addition of Xho I and Mul I sites in recombination primers

Xho I
ctcGAGTCAGTGTCTGGCAGTGAATGAGCTACAAATTGTTTTCATATTGC
TTACCTGAAGGCCAGTGCTTGTTTAGCTGCTGAAGAAAAATAGAAACCTT
ATGGCATTTAGAACATAGTTTATTCTTTAAGTGCAGAAGTGTGTGACTTA
ACCCTTGACTGGCATGGTCTTAGCTCCTGTTTACAATTTGGTATCTTACT
GCCACAAAGAGTCTGTTCTATCAGTCTTACATTCTCTATTTTCACATCAA
TGCTGGCCAGTTGTGTCTAAACACTGCAAAAGGGAGGGTATATAACAAGA
TATGTCTGACTTCCTGAGCCATCATGGCTGGGAACTCAGTTTTTAAGATT
TATCTAGGGTCCATTTGGCCAAGATTGGGGTGGGGAGGGTCTGTTCAGTC
AGTTGAGGGCTTTAAGATTTATTTTTAGTTTACAGGAGGAACCATGTTCA
GACTACCAAATAGTTTCCAATGCTGAGCAGGGCTAGGGCCACAGACACAC
ACTCCTGTTTCCATAGAAATTCCTCAGGCAGGCCTCATGTACTTAGCCAG
GACCCTGCTCTTGCCCCCAAAGGAATTTATCCTTTTAAAAGTATAATATT
CCTCTTATCTGACTGTTCTACTGCACAGTGGGGTAACCACAGTTAACAAT
ATCATATACTATATTTCAAAATAGCTATTAATAGAAGAGAGAATTTTGAA
TATTCTCACCATAAAGAAATGACAAATGTTTGAAGTGATTGATATGCTAA
TTACCCTGATTTGATCATTACATAATGTAAACATGTATAGAAACATCACT
TAAATATAAACATGCACAATTACTAGATGTCAGTTAAAAACAAAATAAAA
CTTAGAAAAGTATAATGTGATGATACTGGTTCAGGGATCTGATCTTTAAT
CCGAAGGAAAAGAGGAGACATGAAAGAACTGAGGGGAGGGCTTGTTATTC
CTCTTCCTCCCCTTTCTCTATTACCACCCTTGCATTAACATGCCCCTGGT
TATCTTGCTATGTTTTTTATATTTATTATGAATGCAGAACACAGCAAAAA
GTAAATAGCAATTATATCAGTTCTAATTCTGCTTCAAGTAATAGAACAAC
TCAAAGCTTATTAAATTAAAGGGAGAATGTATTGGTTCACGTAATTGAAA
AGTCTTGAAATGGGGCTAGCAGGCTGTACTCACTCCAAGGCTCAAAGTAT
ATCATCAAGATTGGGTGAGTCTCTCAATCTCTCAGCTCTGCTTTTCCCTC
TGTTGACTACAGTTTTGACATGTAGTCATGAGATGGCTGCAGCAGCTTGG
CCTACATTCTTCCCTGTTCAAATCTCAAGGTGCAGGGGCCAAGGGGTTGG
GGGGAGAGGTGGAGGAGGAATGCTATGTCTTTATTGGTAGTTCACACAAT
AGTCCTGAGATTCACTCTGCCTTAGACATCCCAGGTCACATGCTTACCCA
TCCCTGTCCACTAACATCCAAGCAACATCTTCACCCTCATAAGTCAAAGA
TAAACTCACCCCCTCACCCTGACCACAAGGAGTAAGAATGGGCAATGGTT
GGATTCTTAAATGAAAATCAGGGACTGTTGCCAGAAAAAAAAAAAAGTGG
AATGTATACAATGGAGGAAAACAATAAATGTACTAGAGCAATTTTTGCTA
CTTTTTTTGTAGTATTTGTATTCAACATTCAATATTCTCCTGAAGCTACT
CTTTTCCTAAAACAGATGAAAACCTTCTCCCCGATCTTCTAGAGATGATC
ACACTGAACCCTAAAGGACATTTAAAAACCTCCATAATCACACAGAGAGG
GACTCAATTAAAAAAAATTCTTGGAAAAGAAAAAGGAAAAGATGTATGTT
TCTTGCTCTTCTCTCTTGATGGAAACAAACACACATGCAAAAATTCAATT
GCAATAAGTGTCTCATATTGAGTTTACCAATTCTGTAAGACTTCATAGTT
ATATAAAACTAATAATTACCATTTACTGAATACCTTGCTATCATGCTAGG
CAAAGTGTCAGACCATTGACATATAATTCATTTAATATACTGAAGAACTA
TCAAAATGAAGTATTATTATCTTTACTCTATAGATGAGGAAACAGACCCA
GAAAGGTTAAGAAATTTGCTCAAAGTCATAGAGCTGGCAACTGGAGGAGC
CAGTAGTCAAAGGCAAGTCTGTGAGGTGTGTGAGATTGTGCTCTCAACAC
TACACCATGGATTTTGTGATCTTGGCTCTAATTAGGGGGCCAATATGGTG
GACATGGTGTTATTTGAATTTAACCATTTTTCATATCTCATATGAAAAGT
TCCTAGAATTAAAAATTTCATGAAGAAAACAAAAAATAGCTGAGGATTTC
TAGATGCATACTGTGAAAAGAAATGCAAATATTAATAGTAACCATGTTCT
ATGAATTAGTTTAAGCATATACATGAAATGCTGTTAGAGTACACGTTGCA
AGAGTAACTAACTGATGGCCTGATTAGAAAAATGATAGAATGAGGTAAAG
AAAGAGGTAGATTCTGGGAAATATGGATTAAAATGATGAAACAAAAAATG
AGAAAAAAACTACCTTACCTTCTCAAGTATTGCCATAGAAGAGCAGAAGT
CATCCTGAGCAAAGCTAAAGGCAAAAAACCTAGGCTCCAGACTGATACAC
TGGCTTGATAATCCACACTGTGAGATGTCACCAAAAGTTGTCTGATCCAG
CTGTGGGGACTTGGCTCCAAGTTGTCTGGATATCCTAAAATAATATAAAT
CCCTTGAAACAAGTGCTCTAAGAAATGGAGGAAGCCATACTTTAGGGAAT
GCTGATAAAATGAGTGGAAATGTTGGTGGTGTGAGTCCACCTCTACCTCA
ACAAGGTCACCACTGATGTGAGAATCCATGAGGTTAACAAAAACGAGCAT
CATTAGTCTCATAAATTAACACTTGAAGGACTTTGGAGCCTTGGGTCTCA
GTGATTCTTCTGGAAATATGGTAGTTTAGGGTCTTTAATAATTTTTTTAA
CTTTTGTTATGAGCAGAAATTGTAAATAACATGTAAATACAACTCCAGAG
TGGTTCTGATGTTCACCTTCTGAACTCATCTAGTTCTCAAACTACTAGAT
TATGCAACCCTTGTGCTCCTACAGCTAAACCACATATACTATTCTGAGCC
CTCCCCTAACTACACCTGGGTAAGAGGAGTGTGGTGTAGTCTGGTGTTGT
GCTATACCAATAGTCATGGAGATATCAATCAAACATCTGCCTAATCTACA
AGGTGTAGGTGGTGAATTATGAAGAATGAGTAACCAGGAGACTTACGATG
AGAGAATAAAGTCTAAGTTGATAAAAAGTTAGTGCTGAGTAGATAGAGGG
GTCTTCAGGAATTGGAAGCTTGGTGAGGGTCCCCATCATCCAGAATAAGT
ACTGGTCTTCAGCAGGAAGAATTTAAGAATAGAAGTTACCTAAAGCTCAT
GAATTAGTATCAGTTCAATTATGCTGGTAATCTGATTTTGATCTTTCTCA
GTTATGATAAAAGTCTCTGCCTGGTCATGATATAAACCTTCTTTGTTCTT
TAGTCTTCCCTACTTACAAGATTAAAGTCCTGGCCTAAAATATGAGCCCT
AAGGTCCTGCTACCTAGTGACATGATATTGTTGATACCAACTCTCCATTA
CCCACTGCCTACTGAAATCTCCTATTCCTGCCTTCTCCATCATTGAGTTC
TGTCCACCTGTCAGAGTCTACTTCATTGGGTTGCCCTCCCTAACCTGCCC
AACACCAACTGCTTGCCAGGCCGCTAACCTATTCCCTCTTGTTCTGCACC
TTCTCTCATAGTCCACTTCCAATGCATCACCAAGATCTGCCTCTAAAATG
TATCTTCTCTTCAATCCCACTGCCAATGCCTTAGTTTGAGATTTTACTAT
CTCTTCCTTCTTTTATATAAATTGCCTGTATCCAGCTCAATCCGCCATCC
ATACTTCCAGTAGTTACATTTACTGAATGAAAATCTGGTCCTGTCACTTT
TCCACTTAAAAACCCTTCAGTTGCCACCCAATATAAATAATATATACATC
ACTTAGAGGCTTCCAAGGCTTCCAAGCCCTTTCCATATGACCTTTCAGGC
CTGCCTCTCCCTGATCTTTCCCATACAATCCAATAAGTGCTTTTATTGTT
CTTTAATGTTCTTTAATGTCCTTTAATGTTCAGCTCAAATATTACCTCCC
CTTGACCTCTCTAAAAGAAAACAGCTTGCTTCCTCAAGTAAAGCTATTTA
CTAATACCTAAGGTATTCCTAAAGGTATTTACAAATACCTTTATTATAAC
ACATATCATACTGTTTATATGTATGCTTTCTTCATTAGATTATAAACTCT
TGGAGGACAAGGAGCATGCCTTCTTCATTTACAGTTCTGTGACAAGCATG
GTGACTGGCAAGTAGTCACTGGCAAGAAATGTTTCTTGAATGAATAAATG
ATCCCTAAATACTGTGACCTATCTCTTAGTCTGAATTTCCCTCAGTTACC
TGCAGAAATTTTCCCTCTGGAATATATTCTTGTTTATCTATCTGTCTACC
TGTCTGTCTGTCTGTCTATTCTATCTATCTATAATCTCCCTATATACAAA
GGAAACAGGTGAGAAAGGAGAGTAGAAGCTTATTTCAAGTTCCAGTCCCT
CCTGATCTACATTCTCCTGTAATTATTAGCCTATGTTACCATGTCTGAAC
AGAAAATATTGGTGATGCCCTTGATCATGAATAGCTATCATGTCTCTGTT
CTGGCTTGCTCCTGGATTTTTTTTTTTCCAGTTTCATGATGCTGCATCAC
TCTGTCACCAGTCCTCACTGTTCGTTTCTATGGAACATGAAATGGTGAAT
GGTCCATCCTTTACTGCTGATACTAGTCATTGCTGAAACAGCCACCCTAA
AGATCTCACAGTCTCTCTGTTTATAAACATTTAAGAGTCAAGTCATGAAG
GCTTTCTCCCTTAACTACATGGGATGATCAGTTGTTTTATCTGTTATTTT
TTCTGTTATTACTTTTGTCCTTAATTGTTAGAGAAAACTTTCATATAACA
CAGTTACTACATAAATGCCCCCTCCCTTCACATCTTAGAATGCCTCTGGC
CATCTCTGTAGTTGTACTTGAGAGGTTTAATAAAGTACCATAAGTTTGGG
AAAATTAGCTTTATTGATATAAGCACTTCCTGGAGAATATTTCTTCACTA
TACAAAAAGCTCATATTTGATCATTGTTTTCTCTATATCTGTCTATCTCT
CTCACATACATTCTGAGTGCCTGTGTGTGTGTGTGTGTCCCCAGTGACTT
CTGCATCTGTAGTAGCTGGAATAACATGTGTGTCACATGTACCCCCACCA
CACACACACACACACACACACCTCTGCCACGGGCTTCGTTCAATTCTATT
CTCAATTTCCAACCTTGTCAGCCATCTTGATTTCCTCTTCTCCAAACCTC
AAAGAAACTGTTAATGAGTACCCGAGAATGAAAATGTTGGGCATATGGAA
AAACTGAAAGACAATCCATATTGCCATAAAATGGCCTGCTTTTATCTGGA
AAAGCCTTATTATCACTCACACTCATTCCTTCTACATCCTTTACCTCCTT
CTCTTTGTCTTTCTGTCTGTCTCTCATTCTGTCTCTATCACACACACACA
CACAACACACACAGAGAGAGAGAGAGGGAGAGAGAGAGAGAGAGAGAGAG
AAAGAGAGACTGGCTATAAAGAAAGCAGCATCTGAGGGCATCAATGGTAA
TTCGAACATTTTGTTTGCTTGGGATCAAGATTCCTTTCCATCACCATTGT
CCTGGCAAATATAAGCTGCAAGTGGAAGTGTTTTAGCCAGATACTGCTCC
ACCACTCTATGGCTAACTAAGCAGGAATATCAGCTTCAACTATGCCATGG
AATTCAAGAGAATATTCAACCTGGAAAAATTCTAACCCCAAACAGCACCT
CCCAAAAGATGACTACAGCTCCTGTAGGAAATCATTTAACCACAAATTCC
AACTCCCCTTCACTCCTACAGCCTCAGTCACACATCTCAAAGGGCTGATC
CTTGAATTGTGACAACCTGACCCACATCAGCAGCCCAGAGGCCAGATGGC
AACACAATGCCTTTCCAGCCTACTGGGTAGGAAAAGGGAGGGACAAGCAA
TTGAATGATTATAATTGAAATCCTGTAATTTATTATTTGTCAATAACTCC
TGCCTTGGGGGAGTTCCCTTCACTCCTTAGCAAATGCTGGCAGCCGCAAT
ATGTGACCACAGACACCTAAAACACCACTGAAAGCATTTCATTATGTGGC
AACAATGATATGGAGAATAAGATCTCTCTGGAGATAAGAAGACATGCCAC
ATCTCAGAGTTCACTATTCAACAGCAAAGAATTTGAATAGGGGGAAAAAT
TCCTTAGACTTAGGGGGAAAAATTCCTGAGATTCTGAGGGTAAAAAGCTA
GCATGCAAGTGGGATCAGCCAGACTGGCAGGAAGTGGGGCATGAAAACCC
AAGAACTATCCTCCTGTTTGCAGTATAATGTTACCCCTGCAGTTATTAAA
CTGCACAGATCAAATAACTATTTGAACTGAGCTGAGCTGAGTCAAGCAAA
GAAATGTACTTCAATCATAGAATAGTAGCTGTCAGGTTGAAAAGGGCATC
TATCAGACATCTACCCAATGTTCCTCTACCTAACTCCCATCCTAATTATC
CCTGTCTCATGTTCATCTCCCTGTAATTGATGCAACCTGATGCAACATAA
AGAGGATTTGGAGTGAAAAGCCCTGAGTTGGAGGCCTGGGCCTTGACTTT
TTAATTTACTAGCCATACCACATTGGTCCTTCTAGTCTTCAGCTACAATG
TGAGCAGATTAAACTAGTTGATGCCAAGATTCCATCCAGTTCAAAATTCT
CTAGGCAATAATAGGGAACTCACTATCTCCAAAAATAATTATTTGCATGT
TTGGACAATTCTATTAAAAAGTCGTCACTTACATAAAGCCAAAACATGGT
TTCTTATAGTTTCTACATATTTATCTGAGTTCTACCTGCCTTGAGATTAT
ATAGTCCAAGTATAATCCCTCTTCTATATAACAGCCACTCATATAGCCAA
AGACAGATATTGCATCTCCCATCCATCTATCCATCCATCCATCCATCCAT
CCATCCCACTGAGTTCTGTTTGCCACTCATCTAGACAATCATTCTATCTA
CTTTACTCAGAACACATACCAATTTGTCCATATCCTTACTGAAAAATGGT
CATCAGAACTGAACAAAGAGCTACAGAAATAGTTTGATAAATGCTGAGCC
AAGTACCTCACCACCTATTTTGTTCCAGCTAATGTGTTTATTTTAATGCA
GCCTAATATCACATTATCTTTTTGGCTATGGCATCACTCTGCCACTGCCA
CAATATTGCTTTTTTTACTCATGTCTTCTACCCTATCCTTGTAAAGTGTA
ATTTGGAACTGGAGTCAGAATTTTACTTTTTCAATAATATTCATTTATCA
GATTTGAGCCATTGGTGCAACATGATAAAATCTTCTTGAATCCTCAGTCT
ATCCTAAAAATATTTGCCATCTATCCAAATTTTCACCTAAGTTATTGATA
AAATACAATGAGCACAACATGGTCAGGAAAAAAGTCCTAGAGCAGACCAC
TAAACAATACCCTCCATATTGACATAATTCTGCAAGATTTGGGGTCTTTT
GGCCCCAAAGAAGGTCATATCCAAGATTGCCTCATTCTTGACACTCTCTC
AATGCTACCTTCTTCACTCACTCATAGTCCATGGTCTAGTCTTTCTGCTG
ATCCTGCAATGATTTCCACAGTTAAGTTCACCCAGACATAGGTACTTTGA
GAGAAATCACTGTTTAAAACATTAAAAATATAAATAAGGTTAACAAAAAA
AGGATAAAAGTTCACACCCAGAACAATATGCCATGGTGGAAATAATGTGG
GAGTTGGAGTCTGAATAACCTCAGATTAAATCCTAGTTCTGTAACTTACT
TGCTTGGTGACCTTGGGTGCCTTATCAGTATCAGTTCCCTCATCTCTAAA
ATAGAGGAATAATAATGACCTCAAAGGGTTGTCCTGATGATTAAAATTAA
TACACGCAAAATCTTAGCACATTCCTAGCACATAATGGGTATACAATATA
GATTACTATCATTATTACATCATGGTAGAGACAAAAATGCAATAACTACT
GGCATAAAAAAATTAGACCCAAGAAGAAAAAAGGAGAAAATGAATATCTG
TGTTCTACAGATGTTAACCAATACTCCACAAAAGAAAGAATCAATGGTCT
AATAAGGTTAAGAACGAGGACATTAAACACAGTTTTAAAAACTCGGGCAT
ATTTTTATCTCTTTGACTCTGAACAAGATTGGCACACCCTTGCATCCCTT
ATTTGATCATGTCCCAGGCATTGCTGTAGGGCCCAAGGACACAAAGGATG
ACTCAGACCCAGTCAATGCTAGGGTGAGAAGAAACACAGGATTCAAGGCA
CCAGGGGGCAGGGGGCACACATATAAATAAGTAATTAATATAAAATATCA
CCAGTGCTATTGAGAGGTAAATACAGAGTATGTGAGGGAACACTGATAAA
TCTGGAGATGTCAGGTAAGGCTTTATGCAGGAGGTGGTAATTTTGAAGAA
TCTTAAAAGATAAAGAGAAGTTAAGGATGAGGGTCTCTCAAGTCAAGGTA
GTGGAGGATAGCACGCAAAGATTTTGAACAGTAGTGGACCAATATGGGCT
CGTCCAAGAAATATTGCTCATTCTAGAGTGACTTTTCCATGAGATACAGG
TAATAAGAGGGACAATTGAAAAGATACAGATTCATGCCTATTGTAAAGGG
GCTTGTACGTCAGGTAAAAAAGTCTGAAATTCTGCAGGCAAGAAGAAACC
AGCACAGCGAATGACATAAGATGGTAAGCCAGCTTAATAGTAGAGAGAGA
GGCTAGTTACGACACTTTTTTTTCTTTACCAATACCCAATTGAAAGATGG
TCAGGTCCAGAACTAAAATTATAGCACTGGAGGTAGAGAATAGGCCTACA
TAACAAAATCTAAGCCTGAGTATAATTGATACGATGTGACATGAGCATGA
GGGGTTAAGGAAAAGGAGAAATGAAAATGACTTTGTCTAGCTTGAGACAC
CCAGTGTGTGGGGGCATCATTACCAATATATGGGCTTCTGGAGGATAGTA
CATTTTGGTAGGAGGCACAATGTAAGTTCAGTTTTCAACACTATGGATTG
AGATAACCATGGGACATCCATATGGAGATGCAATCTGCTCAACATAACAG
TGTGTATATCATAAGACAGACCAGGGATAGGAGTCCCCTGTATATTTATG
GCAATAAAAGAAAATTCATGGTTTAAGAAGGAAGAGTATGTGGAACATGT
CTCTGATGCCACGATGTAATAACCTTGAAAACAAAGTAAAATTACACTAA
TGAGTCTTGCCTAATTAAACTCATGCTCCTAGTGATCACCACTTCTAGTT
CAATTGTTCACATTCTTGCTCTGCTTTGAAAAATTAAAATTAAATTTGCC
TATCCTCTACTGACCATAATTTCTAGAAGACGGCATTCATCTCATGGCAA
GTTCTTCAGTACCCAAAGATGGAATACATAGATTAAAAAAGAACATATAT
GTAGATGCTTGTGATGTTTTCCTATCATAAATTGAATTTCAAGTTCTTAT
AAACGTATTAATATGTCCTACTCTTCTAGAGACAAGGATCAGGAAGTGTA
TTTATCAATAGATATTTACCAAGCACCTGTCAAGCCAAAGTGGGGTTACA
GAAAAGTAGGTATGGGCCCTGCACACAAACAACCTGTATTAGCCAAAGGG
ACCCTTCCATAAAATTTCCAATATGTAAACCCAAATTTGGAACTTGCTGA
AACAAGTACAGATGAGTACGTGAGGAAGCTGGGAAGTAAACACAGGTTGC
TGGAGAAATAGAGGTGGAGATATGGGTGGATCTAGGTTTGGTTAGGAATG
AATCAGACCATCCCACAGAGGGTGGCTCCTCCCTGCATGGGGCCTGCTAT
AAAAGGGCCATTATCTCAGCCTTCAGTACCCAGCAGGCTCCTTCAGGCTA
CATTCTATTTGCTCTTTTGGTGAACAAGGTAAGAAGGAATACgcgt
Mlu I

FLG Intron Fragment (pGL-intron, clone 243)

Generated by PCR with primers, adding Mlu I and
HinD III sites.
Cloned into pGL4.21 with HinD III and blunt EcoRV.
adg3175′ aggtaagtcacgcgtatcttgtcatatggctaactgg 3′
FLG homology
Mlu I
adg3185′ tca agc ttt tgg caa taa atg tga acc 3′
FLG homology
Hind III
acgcgtatcttgtcatatggctaactggctttcagagaattgtgtcttag
caaattaatcatttattaagttagcttttggggaagtaattttcagtaaa
tttgtataagtaaattttactgcttaaaaccagtcagcctttctaaattc
ttgttgctagcaatggcaagatcagcaagagggatgatcttacctagagc
acactgaggtctgtgagactactaagtccagctgtaagtgggcacaaggc
caaaaaatgggcaattttatttaagtcaggaaaactctattcatttgttt
tatgtagatggtagagaaaagtttctcatgtccatcttccattcttaaag
atggattacttagaggaaaatgtaggtttaaaatgtggaaaatgattgta
ataatataacctagaaaaaaagatgtataaaaaagtgcattttcatttct
tctttctagGTTCACATTTATTGCCAAAAGctt

### Full Promoter Construct:

Xho I-Mlu I fragment cloned into pGL-intron vector
to make full construct driving
luc2p in pGL4.21
Total is 10,623 bp Xho I-HinD III fragment in
pGL4.21
Note nucleotides 1-6 [CTCGAG]: Xho I site
ctcgagtcagtgtctggcagtgaatgagctacaaattgttttcatattgc
ttacctgaaggccagtgcttgtttagctgctgaagaaaaatagaaacctt
atggcatttagaacatagtttattctttaagtgcagaagtgtgtgactta
acccttgactggcatggtcttagctcctgtttacaatttggtatcttact
gccacaaagagtctgttctatcagtcttacattctctattttcacatcaa
tgctggccagttgtgtctaaacactgcaaaagggagggtatataacaaga
tatgtctgacttcctgagccatcatggctgggaactcagtttttaagatt
tatctagggtccatttggccaagattggggtggggagggtctgttcagtc
agttgagggctttaagatttatttttagtttacaggaggaaccatgttca
gactaccaaatagtttccaatgctgagcagggctagggccacagacacac
actcctgtttccatagaaattcctcaggcaggcctcatgtacttagccag
gaccctgctcttgcccccaaaggaatttatccttttaaaagtataatatt
cctcttatctgactgttctactgcacagtggggtaaccacagttaacaat
atcatatactatatttcaaaatagctattaatagaagagagaattttgaa
tattctcaccataaagaaatgacaaatgtttgaagtgattgatatgctaa
ttaccctgatttgatcattacataatgtaaacatgtatagaaacatcact
taaatataaacatgcacaattactagatgtcagttaaaaacaaaataaaa
cttagaaaagtataatgtgatgatactggttcagggatctgatctttaat
ccgaaggaaaagaggagacatgaaagaactgaggggagggcttgttattc
ctcttcctcccctttctctattaccacccttgcattaacatgcccctggt
tatcttgctatgttttttatatttattatgaatgcagaacacagcaaaaa
gtaaatagcaattatatcagttctaattctgcttcaagtaatagaacaac
tcaaagcttattaaattaaagggagaatgtattggttcacgtaattgaaa
agtcttgaaatggggctagcaggctgtactcactccaaggctcaaagtat
atcatcaagattgggtgagtctctcaatctctcagctctgcttttccctc
tgttgactacagttttgacatgtagtcatgagatggctgcagcagcttgg
cctacattcttccctgttcaaatctcaaggtgcaggggccaaggggttgg
ggggagaggtggaggaggaatgctatgtctttattggtagttcacacaat
agtcctgagattcactctgccttagacatcccaggtcacatgcttaccca
tccctgtccactaacatccaagcaacatcttcaccctcataagtcaaaga
taaactcaccccctcaccctgaccacaaggagtaagaatgggcaatggtt
ggattcttaaatgaaaatcagggactgttgccagaaaaaaaaaaaagtgg
aatgtatacaatggaggaaaacaataaatgtactagagcaatttttgcta
ctttttttgtagtatttgtattcaacattcaatattctcctgaagctact
cttttcctaaaacagatgaaaaccttctccccgatcttctagagatgatc
acactgaaccctaaaggacatttaaaaacctccataatcacacagagagg
gactcaattaaaaaaaattcttggaaaagaaaaaggaaaagatgtatgtt
tcttgctcttctctcttgatggaaacaaacacacatgcaaaaattcaatt
gcaataagtgtctcatattgagtttaccaattctgtaagacttcatagtt
atataaaactaataattaccatttactgaataccttgctatcatgctagg
caaagtgtcagaccattgacatataattcatttaatatactgaagaacta
tcaaaatgaagtattattatctttactctatagatgaggaaacagaccca
gaaaggttaagaaatttgctcaaagtcatagagctggcaactggaggagc
cagtagtcaaaggcaagtctgtgaggtgtgtgagattgtgctctcaacac
tacaccatggattttgtgatcttggctctaattagggggccaatatggtg
gacatggtgttatttgaatttaaccatttttcatatctcatatgaaaagt
tcctagaattaaaaatttcatgaagaaaacaaaaaatagctgaggatttc
tagatgcatactgtgaaaagaaatgcaaatattaatagtaaccatgttct
atgaattagtttaagcatatacatgaaatgctgttagagtacacgttgca
agagtaactaactgatggcctgattagaaaaatgatagaatgaggtaaag
aaagaggtagattctgggaaatatggattaaaatgatgaaacaaaaaatg
agaaaaaaactaccttaccttctcaagtattgccatagaagagcagaagt
catcctgagcaaagctaaaggcaaaaaacctaggctccagactgatacac
tggcttgataatccacactgtgagatgtcaccaaaagttgtctgatccag
ctgtggggacttggctccaagttgtctggatatcctaaaataatataaat
cccttgaaacaagtgctctaagaaatggaggaagccatactttagggaat
gctgataaaatgagtggaaatgttggtggtgtgagtccacctctacctca
acaaggtcaccactgatgtgagaatccatgaggttaacaaaaacgagcat
cattagtctcataaattaacacttgaaggactttggagccttgggtctca
gtgattcttctggaaatatggtagtttagggtctttaataatttttttaa
cttttgttatgagcagaaattgtaaataacatgtaaatacaactccagag
tggttctgatgttcaccttctgaactcatctagttctcaaactactagat
tatgcaacccttgtgctcctacagctaaaccacatatactattctgagcc
ctcccctaactacacctgggtaagaggagtgtggtgtagtctggtgttgt
gctataccaatagtcatggagatatcaatcaaacatctgcctaatctaca
aggtgtaggtggtgaattatgaagaatgagtaaccaggagacttacgatg
agagaataaagtctaagttgataaaaagttagtgctgagtagatagaggg
gtcttcaggaattggaagcttggtgagggtccccatcatccagaataagt
actggtcttcagcaggaagaatttaagaatagaagttacctaaagctcat
gaattagtatcagttcaattatgctggtaatctgattttgatctttctca
gttatgataaaagtctctgcctggtcatgatataaaccttctttgttctt
tagtcttccctacttacaagattaaagtcctggcctaaaatatgagccct
aaggtcctgctacctagtgacatgatattgttgataccaactctccatta
cccactgcctactgaaatctcctattcctgccttctccatcattgagttc
tgtccacctgtcagagtctacttcattgggttgccctccctaacctgccc
aacaccaactgcttgccaggccgctaacctattccctcttgttctgcacc
ttctctcatagtccacttccaatgcatcaccaagatctgcctctaaaatg
tatcttctcttcaatcccactgccaatgccttagtttgagattttactat
ctcttccttcttttatataaattgcctgtatccagctcaatccgccatcc
atacttccagtagttacatttactgaatgaaaatctggtcctgtcacttt
tccacttaaaaacccttcagttgccacccaatataaataatatatacatc
acttagaggcttccaaggcttccaagccctttccatatgacctttcaggc
ctgcctctccctgatctttcccatacaatccaataagtgcttttattgtt
ctttaatgttctttaatgtcctttaatgttcagctcaaatattacctccc
cttgacctctctaaaagaaaacagcttgcttcctcaagtaaagctattta
ctaatacctaaggtattcctaaaggtatttacaaatacctttattataac
acatatcatactgtttatatgtatgctttcttcattagattataaactct
tggaggacaaggagcatgccttcttcatttacagttctgtgacaagcatg
gtgactggcaagtagtcactggcaagaaatgtttcttgaatgaataaatg
atccctaaatactgtgacctatctcttagtctgaatttccctcagttacc
tgcagaaattttccctctggaatatattcttgtttatctatctgtctacc
tgtctgtctgtctgtctattctatctatctataatctccctatatacaaa
ggaaacaggtgagaaaggagagtagaagcttatttcaagttccagtccct
cctgatctacattctcctgtaattattagcctatgttaccatgtctgaac
agaaaatattggtgatgcccttgatcatgaatagctatcatgtctctgtt
ctggcttgctcctggatttttttttttccagtttcatgatgctgcatcac
tctgtcaccagtcctcactgttcgtttctatggaacatgaaatggtgaat
ggtccatcctttactgctgatactagtcattgctgaaacagccaccctaa
agatctcacagtctctctgtttataaacatttaagagtcaagtcatgaag
gctttctcccttaactacatgggatgatcagttgttttatctgttatttt
ttctgttattacttttgtccttaattgttagagaaaactttcatataaca
cagttactacataaatgccccctcccttcacatcttagaatgcctctggc
catctctgtagttgtacttgagaggtttaataaagtaccataagtttggg
aaaattagctttattgatataagcacttcctggagaatatttcttcacta
tacaaaaagctcatatttgatcattgttttctctatatctgtctatctct
ctcacatacattctgagtgcctgtgtgtgtgtgtgtgtccccagtgactt
ctgcatctgtagtagctggaataacatgtgtgtcacatgtacccccacca
cacacacacacacacacacacctctgccacgggcttcgttcaattctatt
ctcaatttccaaccttgtcagccatcttgatttcctcttctccaaacctc
aaagaaactgttaatgagtacccgagaatgaaaatgttgggcatatggaa
aaactgaaagacaatccatattgccataaaatggcctgcttttatctgga
aaagccttattatcactcacactcattccttctacatcctttacctcctt
ctctttgtctttctgtctgtctctcattctgtctctatcacacacacaca
cacaacacacacagagagagagagagggagagagagagagagagagagag
aaagagagactggctataaagaaagcagcatctgagggcatcaatggtaa
ttcgaacattttgtttgcttgggatcaagattcctttccatcaccattgt
cctggcaaatataagctgcaagtggaagtgttttagccagatactgctcc
accactctatggctaactaagcaggaatatcagcttcaactatgccatgg
aattcaagagaatattcaacctggaaaaattctaaccccaaacagcacct
cccaaaagatgactacagctcctgtaggaaatcatttaaccacaaattcc
aactccccttcactcctacagcctcagtcacacatctcaaagggctgatc
cttgaattgtgacaacctgacccacatcagcagcccagaggccagatggc
aacacaatgcctttccagcctactgggtaggaaaagggagggacaagcaa
ttgaatgattataattgaaatcctgtaatttattatttgtcaataactcc
tgccttgggggagttcccttcactccttagcaaatgctggcagccgcaat
atgtgaccacagacacctaaaacaccactgaaagcatttcattatgtggc
aacaatgatatggagaataagatctctctggagataagaagacatgccac
atctcagagttcactattcaacagcaaagaatttgaatagggggaaaaat
tccttagacttagggggaaaaattcctgagattctgagggtaaaaagcta
gcatgcaagtgggatcagccagactggcaggaagtggggcatgaaaaccc
aagaactatcctcctgtttgcagtataatgttacccctgcagttattaaa
ctgcacagatcaaataactatttgaactgagctgagctgagtcaagcaaa
gaaatgtacttcaatcatagaatagtagctgtcaggttgaaaagggcatc
tatcagacatctacccaatgttcctctacctaactcccatcctaattatc
cctgtctcatgttcatctccctgtaattgatgcaacctgatgcaacataa
agaggatttggagtgaaaagccctgagttggaggcctgggccttgacttt
ttaatttactagccataccacattggtccttctagtcttcagctacaatg
tgagcagattaaactagttgatgccaagattccatccagttcaaaattct
ctaggcaataatagggaactcactatctccaaaaataattatttgcatgt
ttggacaattctattaaaaagtcgtcacttacataaagccaaaacatggt
ttcttatagtttctacatatttatctgagttctacctgccttgagattat
atagtccaagtataatccctcttctatataacagccactcatatagccaa
agacagatattgcatctcccatccatctatccatccatccatccatccat
ccatcccactgagttctgtttgccactcatctagacaatcattctatcta
ctttactcagaacacataccaatttgtccatatccttactgaaaaatggt
catcagaactgaacaaagagctacagaaatagtttgataaatgctgagcc
aagtacctcaccacctattttgttccagctaatgtgtttattttaatgca
gcctaatatcacattatctttttggctatggcatcactctgccactgcca
caatattgctttttttactcatgtcttctaccctatccttgtaaagtgta
atttggaactggagtcagaattttactttttcaataatattcatttatca
gatttgagccattggtgcaacatgataaaatcttcttgaatcctcagtct
atcctaaaaatatttgccatctatccaaattttcacctaagttattgata
aaatacaatgagcacaacatggtcaggaaaaaagtcctagagcagaccac
taaacaataccctccatattgacataattctgcaagatttggggtctttt
ggccccaaagaaggtcatatccaagattgcctcattcttgacactctctc
aatgctaccttcttcactcactcatagtccatggtctagtctttctgctg
atcctgcaatgatttccacagttaagttcacccagacataggtactttga
gagaaatcactgtttaaaacattaaaaatataaataaggttaacaaaaaa
aggataaaagttcacacccagaacaatatgccatggtggaaataatgtgg
gagttggagtctgaataacctcagattaaatcctagttctgtaacttact
tgcttggtgaccttgggtgccttatcagtatcagttccctcatctctaaa
atagaggaataataatgacctcaaagggttgtcctgatgattaaaattaa
tacacgcaaaatcttagcacattcctagcacataatgggtatacaatata
gattactatcattattacatcatggtagagacaaaaatgcaataactact
ggcataaaaaaattagacccaagaagaaaaaaggagaaaatgaatatctg
tgttctacagatgttaaccaatactccacaaaagaaagaatcaatggtct
aataaggttaagaacgaggacattaaacacagttttaaaaactcgggcat
atttttatctctttgactctgaacaagattggcacacccttgcatccctt
atttgatcatgtcccaggcattgctgtagggcccaaggacacaaaggatg
actcagacccagtcaatgctagggtgagaagaaacacaggattcaaggca
ccagggggcagggggcacacatataaataagtaattaatataaaatatca
ccagtgctattgagaggtaaatacagagtatgtgagggaacactgataaa
tctggagatgtcaggtaaggctttatgcaggaggtggtaattttgaagaa
tcttaaaagataaagagaagttaaggatgagggtctctcaagtcaaggta
gtggaggatagcacgcaaagattttgaacagtagtggaccaatatgggct
cgtccaagaaatattgctcattctagagtgacttttccatgagatacagg
taataagagggacaattgaaaagatacagattcatgcctattgtaaaggg
gcttgtacgtcaggtaaaaaagtctgaaattctgcaggcaagaagaaacc
agcacagcgaatgacataagatggtaagccagcttaatagtagagagaga
ggctagttacgacacttttttttctttaccaatacccaattgaaagatgg
tcaggtccagaactaaaattatagcactggaggtagagaataggcctaca
taacaaaatctaagcctgagtataattgatacgatgtgacatgagcatga
ggggttaaggaaaaggagaaatgaaaatgactttgtctagcttgagacac
ccagtgtgtgggggcatcattaccaatatatgggcttctggaggatagta
cattttggtaggaggcacaatgtaagttcagttttcaacactatggattg
agataaccatgggacatccatatggagatgcaatctgctcaacataacag
tgtgtatatcataagacagaccagggataggagtcccctgtatatttatg
gcaataaaagaaaattcatggtttaagaaggaagagtatgtggaacatgt
ctctgatgccacgatgtaataaccttgaaaacaaagtaaaattacactaa
tgagtcttgcctaattaaactcatgctcctagtgatcaccacttctagtt
caattgttcacattcttgctctgctttgaaaaattaaaattaaatttgcc
tatcctctactgaccataatttctagaagacggcattcatctcatggcaa
gttcttcagtacccaaagatggaatacatagattaaaaaagaacatatat
gtagatgcttgtgatgttttcctatcataaattgaatttcaagttcttat
aaacgtattaatatgtcctactcttctagagacaaggatcaggaagtgta
tttatcaatagatatttaccaagcacctgtcaagccaaagtggggttaca
gaaaagtaggtatgggccctgcacacaaacaacctgtattagccaaaggg
acccttccataaaatttccaatatgtaaacccaaatttggaacttgctga
aacaagtacagatgagtacgtgaggaagctgggaagtaaacacaggttgc
tggagaaatagaggtggagatatgggtggatctaggtttggttaggaatg
aatcagaccatcccacagagggtggctcctccctgcatggggcctgctat
aaaagggccattatctcagccttcagtacccagcaggctccttcaggcta
cattctatttgctCTTTTGGTGAACAAGgtaagaaggaat [symbol] atct
tgtcatatggctaactggctttcagagaattgtgtcttagcaaattaatc
atttattaagttagcttttggggaagtaattttcagtaaatttgtataag
taaattttactgcttaaaaccagtcagcctttctaaattcttgttgctag
caatggcaagatcagcaagagggatgatcttacctagagcacactgaggt
ctgtgagactactaagtccagctgtaagtgggcacaaggccaaaaaatgg
gcaattttatttaagtcaggaaaactctattcatttgttttatgtagatg
gtagagaaaagtttctcatgtccatcttccattcttaaagatggattact
tagaggaaaatgtaggtttaaaatgtggaaaatgattgtaataatataac
ctagaaaaaaagatgtataaaaaagtgcattttcatttcttctttctagG
TTCACATTTATTGCCAAAAGCTT
Bold sequence [CTTTTGGTGAACAAG]: FLG exon 1
(part of 5′UTR)
Bold/italic sequence [ACGCGT]: Mlu I
Underlined sequence [GTTCACATTTATTGCCAAAAG]:
FLG exon 2, (part of 5′UTR)
Highlighted sequence [AAGCTT]: HinD III

### Transgenic Animal Production

The FLG-10k-luc2p construct was sent to TaconicArtemis GmbH under contract to generate a single-copy transgenic mouse via mouse embryonic stem cell gene targeting into the murine ROSA26 Icous. ROSA26 is a site where high-efficiency gene targeting can be achieved and is a locus where at least heterozygous transgene insertion exerts no harmful phenotypic effects. The resultant mice were transported to Dundee.

### Detection of Luciferase (FIG. 4)

Indirect immunofluorescence was used to show co-expression of luciferase with endogenous mouse filaggrin.

### Assessing Test Agents (See FIG. 6)

All mice were treated with a single intradermal injection on day 0 and were imaged daily for 6 days.

### Results

### Live Animal Imaging

FLG-luc2p heterzygous animals were subjected to live animal imaging using the Caliper/Xenogen IVIS200 system, which allows imaging of the luc2p gene bioluminescence signal, following intraperitoneal injection of luciferin (the luc2p substrate), thus revealing, in real-time, quantifiable in vivo luciferase gene activity. Surprisingly, luc2p reporter gene expression in these mice was largely detected in the footpad epidermis, whereas one would have predicted that the FLG promoter would drive expression all over the epidermis. Upon covering the footpads and performing longer exposures, luc2p expression can be detected elsewhere in the epidermis but at a much lower level than footpad.

The results of live animal imaging (under anaesthetic) are shown in FIG. 2. Very strong luciferase expression is detected in the footpad and much lower levels elsewhere in the epidermis. FIG. 2(a) provides a colour bar showing the gene expression range quantified by the IVIS200 (red tones=maximal expression; blue tones=minimal expression). Footpad expression following a 1 second exposure in the IVIS200 is shown in FIG. 2(b). Note that expression is also very strong in the forepaws (covered up here). When all paws are covered, a 60 second exposure reveals expression in the tail epidermis, as well as shaved back, ears and snout (see FIG. 2(c)). Again, when paws are covered, a 60 second exposure reveals expression in the epidermis of tail, shaved stomach, perioral and perianal regions (see FIG. 2d). Thus, reporter gene expression is widespread in the epidermis but is vastly greater in the footpads.

### Regional Expression of Luciferase in the FLG-luc2p Mouse (FIG. 3).

Regional gene expression was also examined by quantitative reverse transcriptase PCR (QRT-PCR; Applied Biosystems Taqman gene expression assay) and western blot for luciferase protein. This confirmed that luc2p expression is very much greater in the footpad epidermis than elsewhere in the mouse epidermis on both the mRNA transcript and protein levels (see FIG. 3, below). By QRT-PCR it was shown that luc2p mRNA expression is much higher in the footpad compared to other epidermal regions. Similarly, FIG. 3b shows that luciferase protein was detected by western blot in footpad tissue but fell below the limits of detection in other epidermal regions. These results are consistent with what was observed by live animal imaging of the bioluminescence signal derived from luc2p expression (see FIG. 2).

### Immunoinfluorescence Detection of Luciferase (FIG. 4)

To determine if the FLG-10K promoter is able to direct expression to the correct epidermal compartment—the granular layer—where filaggrin is expressed, immunofluorescence staining was performed in mouse footpad epidermis using antibodies against murine filaggrin and luciferase. This revealed that indeed, the FLG-10K promoter directs expression to the correct epidermal cell layers (see FIG. 4). FIG. 4(b) shows that the endogenous filaggrin is localised to the outermost living layers of the epidermis (granular layers). In wild-type mouse epidermis FIG. 4c shows that there is no luciferase expression. In the FLG-luc mouse, luciferase is localised to the granular layers, mirroring the expression of endogenous filaggrin (See FIG. 4d and compare to FIG. 4b). Overall, these data show that the FLG-10K promoter, when targeted into the murine ROSA26 locus, drives reporter gene expression in the granular layer of the epidermis and that this expression is largely limited to the footpad—the region of the mouse skin that is most similar histologically to human skin.

### Left-to-Right Ratio Analysis (FIG. 5)

This footpad-localised expression was not predicted and may be a consequence of (a) the precise FLG-10K sequence used including its shortened intron 1 and lack of intron 2; (b) targeting into ROSA26, outside of the epidermal differentiation complex gene cluster where FLG is normally located; and/or (c) the fact that the human filaggrin promoter was used here to direct expression in the mouse.

The footpad-restricted high-level reporter gene expression in the FLG-luc2p mice is particularly useful for analysis of filaggrin gene expression in real-time. The well circumscribed expression in the footpad, with negligible expression elsewhere in the epidermis, makes this mouse model exceptionally well suited to split body studies where one paw receives injected or topical treatment and the other side receives control treatment. For this to be a useful system for testing cutaneous delivery of therapeutics, the left-to-right expression levels must be consistent from day to day. This was investigated by imaging the same set of FLG-luc2p mice over time and comparing quantification of bioluminescence signals from the left and right footpads (FIG. 5). This showed that, although there are clear differences in overall gene expression from day to day (FIG. 5a), the left-to-right ratio is very consistent (FIG. 5b). Therefore, these mice are highly suitable for split body trials designed to assess delivery of drugs or gene silencing agents to the epidermis.

### Assessing Test Agents (FIG. 6)

To test the ability of FLG-luc mice to act as a model for epidermal delivery of drugs and gene silencing agents, siRNA-targeting luc2p was intradermally injected into the left footpad of FLG-luc2p mice. Control siRNA was injected into the right footpad. Two types of siRNA chemistries were used: native RNA or Dharmacon's Accell siRNA chemistry (nuclease resistant with a self-delivery moiety to aid cell penetration). Mice were given a single injection of siRNA on day 0 and were imaged daily over 6 days. The results are summarised in FIG. 6. This analysis showed that (a) these mice are very useful for assessing epidermal delivery of molecules aimed at silencing gene expression; (b) relative efficacy and longevity of gene silencing agents (here Accell was superior to native RNA chemistry) and similarly, (c) small molecules or other agents aimed at up- or down-regulation of filaggrin gene expression can also be assayed in vivo.

PBS treatment only had no effect on left-right ratio over the entire time course; see also green trace FIG. 6b. In the test mice the left paw was injected with either Accell siRNA or native siRNA targeting the luc2p mRNA and the right paw was treated with the corresponding native siRNA or Accell siRNA non-targeting control siRNA (NSC4, an inverted bacterial lacZ sequence). In both cases, the luc2p-specific siRNA knocked down luciferase gene expression. In the case of Accell, gene expression was reduced by 80% and remained at this level for 4 days, after which the left-right ratio returned slowly to 1. In the case of native siRNA, the level of knockdown was only slightly lower but it returned to baseline more quickly.

### Discussion

We have developed a transgenic mouse expressing a luciferase reporter gene under control of a modified human filaggrin gene promoter. This is a very valuable animal model that will allow in vivo analysis and validation of chemical compounds that can up-regulate the activity of the human gene.

Surprisingly, we found that animals modified to harbour the nucleic acid constructs described herein, generated a luciferase reporter gene that is expressed primarily in the footpad epidermis rather than all over the epidermis. Thus, these animals allow live imaging of the luciferase reporter in small, circumscribed areas and left-right comparison in vehicle/control experiments such as assessment of topically applied filaggrin upregulation compounds. Moreover, the mouse footpad epidermis is most comparable to human skin in terms of tissue architecture.

In addition to the study of filaggrin gene regulation, these animals can also be used for the in vivo assessment of cutaneous delivery of gene silencing technologies, such as small interfering RNA (siRNA) or antisense molecules, applied by injection, systemically or topical formulation.

### Summary

FLG-luc mice express the luciferase reporter gene, luc2p, primarily in the footpad, the site of mouse epidermis most similar in structure to human epidermis, with low-level negligible expression elsewhere in the epidermis.

The FLG-10K promoter directs expression to the granular layer of the epidermis, where filaggrin is normally expressed in mouse or human epidermis.

Using left-right ratio analysis, these mice are a useful model system for assessing epidermal delivery, and relative efficacy and longevity of gene silencing agents (e.g. siRNA).

Similarly, these mice can be used to assess cutaneous delivery of small molecules or other agents aimed at modulating filaggrin gene expression, in real-time, in living animals.

1. An isolated nucleic acid, said nucleic acid encoding a filaggrin promoter element and a nucleic acid sequence operatively linked thereto.

2. The isolated nucleic acid sequence of claim 1, wherein the filaggrin promoter element encodes a transcription regulator.

3. The isolated nucleic acid sequence of claim 1, wherein the filaggrin promoter element encodes a human filaggrin gene transcription regulator.

4. The isolated nucleic acid sequence of claim 1, wherein the filaggrin promoter element comprises a sequence exhibiting a degree of identity or homology to a wild type or native filaggrin promoter sequence.

5. The isolated nucleic acid sequence of claim 1, wherein the filaggrin promoter element comprises a sequence selected from the group consisting of:
(i) a sequence exhibiting a degree of identity or homology to SEQ ID NO: 1;
(ii) a promoter fragment having a sequence exhibiting a degree of identity/homology to a fragment or fragments of SEQ ID NO: 1; and/or
(iii) a human filaggrin promoter sequence derived from BAC clone RP1-14N1.2.

6. The isolated nucleic acid of claim 1, wherein the isolated nucleic acid further comprises a human filaggrin gene sequence or one or more fragment(s) or portion(s) thereof.

7. The isolated nucleic acid of claim 6, wherein the human filaggrin gene sequence or the one or more fragment(s) or portion(s) thereof, comprises a sequence exhibiting a degree of identity or homology to a nucleotide sequence of exon 1 of the human filaggrin gene.

8. The isolated nucleic acid sequence of claim 6, wherein the human filaggrin gene sequence comprises or consists of the sequence of SEQ ID NO: 2.

9. The isolated nucleic acid of claim 6, wherein the human filaggrin gene sequence or the one or more fragment(s) or portion(s) thereof, further comprises a sequence exhibiting a degree of identity or homology to 5′ and/or 3′ regions or domains of intron 1 of the human filaggrin gene sequence.

10. The isolated nucleic acid sequence of claim 9, wherein the sequence exhibiting a degree of identity or homology to 5′ and/or 3′ regions or domains of intron 1 of the human filaggrin gene sequence, comprises or consists of SEQ ID NO: 3 or SEQ ID NO: 4

11. The isolated nucleic acid sequence of claim 1, wherein the nucleic acid sequence comprises a filaggrin promoter element and a human filaggrin gene sequence comprising one or more sequences selected from the group consisting of:
a sequence exhibiting a degree of identity or homology to SEQ ID NO: 2 or a fragment thereof;
a sequence exhibiting a degree of identity and/or homology to SEQ ID NO: 3 or a fragment thereof;
a sequence exhibiting a degree of identity and/or homology to SEQ ID NO: 4 or a fragment thereof; and
a sequence exhibiting a degree of identity and/or homology to SEQ ID NO: 5 or a fragment thereof.

12. The isolated nucleic acid sequence of claim 6, wherein the human filaggrin gene sequence comprises, or further comprises a sequence of exon 2 of the human filaggrin gene sequence.

13. The isolated nucleic acid sequence of claim 12, wherein the sequence of exon 2 of the human filaggrin gene sequence comprises a 5′ region sequence or domain sequence of exon 2.

14. The isolated nucleic acid sequence of claim 12, wherein the sequence of exon 2 of the human filaggrin gene comprises or consists of SEQ ID NO: 6.

15. The isolated nucleic acid sequence of claim 1, wherein the nucleic acid sequence comprises or consists or consists essentially of, SEQ ID NO: 7.

16. The isolated nucleic acid sequence of claim 1, wherein the sequence operatively linked to the filaggrin promoter element encodes a reporter gene.

17. The isolated nucleic acid sequence of claim 1 wherein the sequence operatively linked to the human filaggrin promoter sequence does not comprise a sequence encoding a functional, complete or wild-type/native human filaggrin gene.

18. The isolated nucleic acid sequence of claim 12, wherein reading 5′ to 3′, the isolated nucleic acid comprises a human filaggrin promoter sequence; a human filaggrin gene sequence and a nucleic acid sequence operatively linked to the human filaggrin promoter sequence.

19. A non-human transgenic animal comprising a nucleic acid sequence according to claim 1.

20. (canceled)

21. (canceled)

22. A method of identifying an agent for use in medicine, said method comprising: contacting a non-human transgenic animal comprising a nucleic acid sequence encoding a filaggrin promoter element and a nucleic acid sequence operatively linked thereto with an agent to be tested; identifying modulation of reporter gene expression; wherein modulation of reporter gene expression indicates that the test agent might be useful in the treatment of a disease.

23. A vector encoding the isolated nucleic acid of claim 1.

24. (canceled)

25. The non-human transgenic animal of claim 19, wherein the non-human transgenic animal is a transgenic mouse comprising cells in which the genome material has being modified such that the ROSA26 locus comprises the nucleic acid sequence according to claim 1.
